(19) United States
(12) Patent Application Publication
(10) Pub. No.: US 2025/0195083 A1
(43) Pub. Date: Jun. 19, 2025

(54) OPERATION EQUIPMENT FOR KNEE SURGERY, OPERATING ROOM PROVIDED WITH SUCH EQUIPMENT, ASSEMBLY KIT, AND CORRESPONDING METHODS FOR ASSEMBLY, OPERATION AND USE

(71) Applicant: Georges SIOUFI, Montreal, Quebec (CA)

(72) Inventor: Georges SIOUFI, Montreal, Quebec (CA)

(21) Appl. No.: 18/694,311

(22) PCT Filed: Sep. 21, 2022
(86) PCT No.: PCT/CA2022/051405

§ 371 Date: Feb. 19, 2025

(30) Foreign Application Priority Data
Sep. 21, 2021 (CA) 3131443

(51) Int. Cl. A61B 17/15; A61B 17/16; A61B 17/68; A61B 17/82; A61B 17/86; A61B 17/88; A61B 90/00

(52) U.S. Cl. CPC A61B 17/158; A61B 17/1604; A61B 17/1637; A61B 17/1677; A61B 17/683; A61B 17/82; A61B 17/8685; A61B 17/8866; A61B 2090/061

(57) ABSTRACT

Disclosed is an innovation in the manner of surgically exposing the knee during total or partial knee arthroplasty, because currently, and as it has always been the case, the conventional surgical approach for the knee used the anterior route but also the medial route, where it was necessary to sever the whole tissue attachment of the inner bone border thereof, said tissue attachment comprising the capsule (envelope), the medial patellofemoral ligament and the tendon of the vastus medialis oblique (muscle) of the quadriceps. The present invention relates in particular to operating equipment intended to facilitate and/or implement a new and innovative surgical approach.

### CROSS REFERENCE TO RELATED APPLICATIONS

This application is a National Stage Application of PCT/CA2022/051405, filed Sep. 21, 2022, which claims benefit of priority to Application No. 3131443, filed Sep. 21, 2021, in Canada, and which applications are incorporated herein by reference. To the extent appropriate, a claim of priority is made to each of the above-disclosed applications.

### FIELD OF THE INVENTION

The present invention relates to the field of surgeries, such as “knee” surgery, for example, and/or any other related type of surgery. More particularly, the present invention relates to operating equipment for innovative knee surgery, for example, and also relates to a system and/or operating room provided with such equipment, as well as to a kit for assembling it (them), and to corresponding methods of assembly, operation and/or use.

### PRIOR ART

According to the reviewed literature, despite rigorous application of all the known rules of the art for this technique, approximately 15% of the results of total knee replacement are unsatisfactory and the causes still remain unknown. These result in chronic pain that is accompanied by limitations in the activities of daily living; especially those that involve going up and down stairs, among other things. Despite the acquisition of a better mastery of the manufacture of prosthesis models, the design of which has evolved considerably over the last three decades to increasingly respect the anatomy and mechanics of the knee; all these elements could not contribute to improve the above numbers. This misunderstanding of the disappointing results has also earned the total knee prosthesis the expression of the “missing link” as opposed to the best results encountered with the total hip prosthesis over the past 30 years, the latter having experienced solutions to several identified problems.

The proper understanding of this problem and the convictions of the present Applicant/inventor are based on his research, his analyses, his clinical observations, the interpretation of his results, his continuous questioning, simulations as well as his personal experience, in particular, as as a medical doctor and orthopedic surgeon for 29 years. These led the Applicant/inventor to explain that the origin of these poor results is the result of a change in the behavior of the patella caused, by the very surgical approach to the knee, considered universal by the orthopedic community. In the past, this surgical approach had been described and practiced for all kinds of knee surgery, long before the advent of prosthetic knee replacement surgeries. This was at a time when immobilization of an operated knee was part of the norm. The Applicant/inventor explains this anomaly by the fact that instead of the surgical approach being adapted to prosthetic knee replacement surgery, the opposite phenomenon, almost natural, would have occurred.

Indeed, the surgical approach to the knee, never having been questioned in the past or had an alternative for comparison, has at its base, according to the Applicant/inventor, fundamental disadvantages which are in opposition to the principles basic knowledge of surgery and tissue healing, including:
1) This surgical approach, although classic (see diagram #2), creates an irreversible weakening of the capsular wall, ligaments and tendon once severed and following their healing, resulting in a permanent imbalance in the alignment of the patella. This weakening is all the more maintained, exaggerated and favored by the need for immediate and rapid mobility of the knee postoperatively, and this well before the end of healing; at best, healing occurs with some elongation of the aforementioned tissues;
2) Soft tissue healing is complete approximately 42 days after surgery, and it takes even longer to mature, which is a paradox compared to the need to move the knee immediately after replacement with a prosthesis. The completion of this healing is theoretically necessary so that the tensile force of the tissues is great enough to resist against the application of a deforming force exerted by the mobilization of the knee especially beyond 90 degrees of flexion during rehabilitation. On the other hand, we know that in reality, if an immobilization of the knee is imposed to promote quality healing, such a condition can only lead to an undesirable and permanent ankylosis of the knee which is incompatible with normal function, the main reason for which it is essential to begin rehabilitation as early as possible, that is to say the day after the operation and sometimes the same day;
3) Also, and by comparison, we know that there are several similar musculoskeletal disorders which normally require immobilization to ensure adequate healing, in order to obtain the desired functional result. Let us quote as examples: the flexor tendon of the finger, the ligaments of the elbow, the tendon of the biceps as well as the tendon of the shoulder, etc., to name only a few which are among many others;
4) Among the tissues sectioned in this anterior surgical approach to the knee, let us also mention, at the microscopic level, the nerve endings specialized in the detection of changes in position (proprioceptors), pressure of the knee (mechanoceptors) as well as pain (nociceptors), which are distributed at the level of the capsule, tendons and ligaments. These nerve endings, once severed, become pain producing due to the trauma suffered directly at the level of the micro-nerve endings but also secondary to the surgical remodeling and local edema at the very site of the repair. In turn, these specialized nerve endings are excited by the change in tissue tension induced by the back-and-forth movement of the knee and by intra-articular pressure increased by the accumulation of excess fluid; wouldn't these surgical traumas at the scale of the specialized endings mentioned above be specifically responsible, at least in part, for the inhibition of the myoneural plate of the vastus medialis oblique of the quadriceps observed during the initial period of four weeks, which follows immediate knee replacement?;
5) The immediate postoperative consequences of a total knee prosthesis involve the arrival of an increased volume of fluid in the knee capsule caused by bleeding. Since the intra-articular space available is limited; this new fluid volume, the production of synovial fluid and the intense inflammatory reaction in response to the surgical insult, contribute greatly to an increase in intra-articular pressure of the knee. In turn, this hyperpressure is an important cause of pain by stimulation of mechanoceptors among others at the level of the capsule;
6) This mechanism, in parallel with the need to mobilize the knee as quickly as possible and beyond 90 degrees of flexion, becomes one of the most formidable experiences of this surgery for the patient and contributes to clearly limiting flexion. This excess volume cannot be expelled anywhere other than the sealed knee cavity;
7) The sutures of the tissues performed during the closure, must consequently and in turn resist the pressure which will be induced during the flexion of the knee in which a surplus volume of reaction liquid has been filled. According to cadaver observations, if the pressure in question exceeds the resisting capacity of the suture threads, or the elasticity of the tissues that the threads hold together, there will be a loss of the tightness of the closure. The latter is first observed around the edge of the suture and not between the close edges, and there is a production of flow of liquid content from the knee, resulting in the formation of an extra-capsular hematoma, which in turn increases the risk of knee infection. All this exacerbates once again the pain encountered in the majority of patients after this surgery, by stimulating the nerve pressure receptors among others;
8) Another type of tissue severed during the classic exposure of the knee are the arterioles of the vascular network which supply blood to the tissues. The resulting vascular damage by reduced blood supply to the area of repair and knee closure is a significant factor and must be taken into consideration. In fact, it is known that a fundamental condition for good healing of cuts is precisely the quality and abundance of vascularization. An area adjacent to the incision may become hypovascularized and secondarily contribute to delayed tissue healing. Another important element that would influence also the vascular flow of the tissues at the very site of the incision is the tension applied to the stitches during closure and the reapproximation of the severed elements during the anterior approach to the knee. The fragile paradox in which the surgeon is confronted is the following: on the one hand, a repair with too tight sutures would result in a reduction of the vascularization, which creates an ischemic zone and consequently a healing of poor quality, or less, retarded. On the other hand, closing with loose sutures with the aim of avoiding the above-mentioned situation would result in an increased risk of a loss of tightness of the knee capsule (envelope) with the formation of excessive outflows, which would increase the risk of infection. Obviously, the exact optimal closing tension for each patient depending on the quality of their tissues, the severity of the correction and their age remains an unknown variable;
9) Since the aforementioned soft tissue structures are not always clearly visible and individualized during closure, it is common that the perfect symmetry of alignment across the wound and closure of these elements are not achievable, even if the knee is placed in flexion during the closure. This results in an asymmetric distribution of tension on the tissues during active flexion and extension(s). This asymmetry may be responsible, at least in part, for joint stiffness;
10) The risk of detachment of the vastus medialis during passive, excessive and early flexion of the knee can cause permanent weakening of the vastus medialis oblique (VMO). This can lead secondarily to an imbalance in the alignment of the patella, very often exacerbated during the internal rotation of the femur in flexion of the knee;
11) The always limited exposure in some patients of the external compartment during the intervention requires a widening of the incision in the conventional approach, the resection of the fat pad, and a 180-degree eversion of the patella. This places excessive tension on the surrounding tissues, further increased by the use of retractors during the surgical procedure. In addition, this increases the risk of iatrogenic damage to the internal collateral ligament, or the use of the lateral approach if the medical approach proves insufficient; and
12) When closing the capsule of the knee, at the end of the intervention, at the level of its tibial attachment distally, we frequently encounter a tissue deficit due to the lower quality at this precise level of the capsule which is often stretched and thinned. The leaky closure explains the presence of the flow of intra-articular content often encountered at the distal end of the wound, typically the day after the operation. The other factor that contributes to the tissue deficit allowing a leaky closure and the fact that patients at the time of undergoing their knee replacement by prosthesis have at the same time a correction of the axis of the knee deformed by osteoarthritis. Thus, the internal deviation of the leg is straightened to reach a normal axis, which creates tension at the level of the medial capsular lip and makes it less mobilizable and available for quality suturing during closure.

For all the disadvantages of the aforementioned conventional surgical approach, and as a result, it would therefore be greatly advantageous to find a new surgical technique/approach and/or corresponding operating equipment for surgical intervention, on a knee, for example, and to be able to overcome and/or reduce/minimize some of the aforementioned drawbacks and disadvantages, if not all of them, and/or at least to be able to do so more in a quicker, simpler, more accurate, more efficient, more economical, more reliable, more tunable, more versatile, more adaptable, more durable, more desirable and/or improved manner, than is possible with the current way of doing things. One can only imagine what it would mean for the patient as well as the third-party payer(s) to be able to initiate an easier, less painful, less expensive rehabilitation, and consequently, a shorter hospital stay, and a return to life activities, faster, and with a higher satisfaction rate.

The question remains: what is the alternative way of exposing the knee properly to allow the application, according to the standards, of the total knee replacement technique, its revision, or others, without compromising the results, and while controlling precisely the return of the normal anatomy of the patella as well as its original mechanical behavior, and this, in all patients without exception? In other words, how to achieve a predictable and reproducible result in all patients?

### SUMMARY OF THE INVENTION

An object of the present invention is therefore to provide operating equipment (and/or a system and/or an operating room comprising such equipment, etc.) which, thanks to its design and its components, would satisfy some of the aforementioned needs and would therefore be an improvement over other related ways, systems and/or methods known in the prior art.

The aforementioned object is achieved, as will be better understood hereinafter, by operating equipment (and/or a system and/or an operating room comprising such equipment, etc.) such as that briefly described in the present description, and such as that exemplified in the accompanying figures.

More particularly, an object of the present invention is to provide an operating equipment comprising at least one piece of equipment being positionable, shaped and sized to cooperate with a joint of a patient intended to be operated on, so to allow a trans-patellar bone and tendon surgical intervention with longitudinal sectioning on the patient's joint, via said at least one piece of equipment of the operating equipment.

According to a possible embodiment of the present invention, the piece of equipment (ex. a series of tools and/or accessories, etc.) of the operating equipment is chosen from a group consisting of a sectioning guide, a sectioning tool, a connection system, a first threaded head, a second threaded head, a connection rod, at least one router bit (ex. one for each threaded head, etc.), a measuring tool, a retention mechanism, a screwable ring, a rotating tool and a grinding tool, for example.

According to another possible embodiment of the present invention, the latter also relates to operating equipment, including a series of corresponding new tools and/or accessories, including, for example: a patellar cutting guide, as well as a drill guide for screw or clamp(s), a patella separator, and a choice of implants for patella fixation. This could in particular comprise an axial compression screw system, a screw system screwed through screwed washers, a system of double U-shaped clamps with stops, a system of cables for crimped strapping, and/or the use of a screw-running ring.

Indeed, such operating equipment allows in particular, and advantageously, a new surgical approach proposed hereby, either trans-patellar bone and tendon with longitudinal cut, which could potentially remedy all the drawbacks encountered with the conventional surgery approach for knee arthroplasty, and which would most likely be responsible for the poor results of conventional surgery.

According to another aspect of the present invention, it also relates to a corresponding system and/or operating room provided with the aforementioned equipment.

According to another aspect of the present invention, it also relates to a kit with corresponding components for assembling the aforementioned equipment, system and/or operating room.

According to another aspect of the present invention, it also relates to a method of assembling components of the aforementioned equipment, system and/or operating room.

According to another aspect of the present invention, it also relates to a method of operating the aforementioned equipment, system and/or operating room, and/or certain components of these.

According to another aspect of the present invention, it also relates to a method of using the aforementioned equipment, system and/or operating room, and/or certain components of these.

According to another aspect of the present invention, it also relates to a set of components to be interchanged on the aforementioned equipment, system and/or operating room.

According to another aspect of the present invention, it also relates to a method of manufacturing one or other of the component(s) of the aforementioned equipment, system and/or operating room.

According to another aspect of the present invention, it also relates to a knee having been operated on with the aforementioned equipment, system, operating room, kit, and/or method(s).

According to another aspect of the present invention, it also relates to a method of doing business with the equipment, system, operating room, kit, method(s), game and/or or the aforementioned product(s).

The objects, advantages, and other characteristics of the present invention will become more apparent on reading the non-limiting description which follows of the preferred embodiments of the invention, shown in the attached drawings, and given by way of example only.

### BRIEF DESCRIPTION OF THE DRAWINGS

FIG. 1 is an illustration of the anatomy of a knee with peri-patellar tissue attachments.

FIG. 2 is a representation of a conventional surgical approach in knee arthroplasty.

FIGS. 3-61 illustrate various components, features, variations and/or possible combinations, of various aspects of the operating equipment of the present invention, according to various possible embodiments.

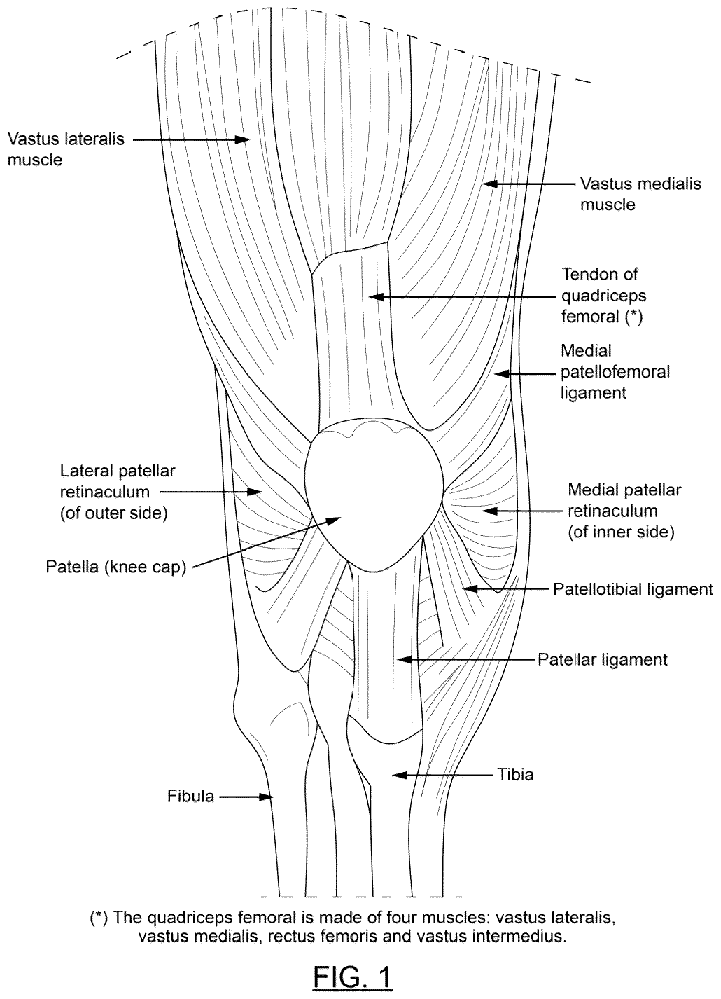

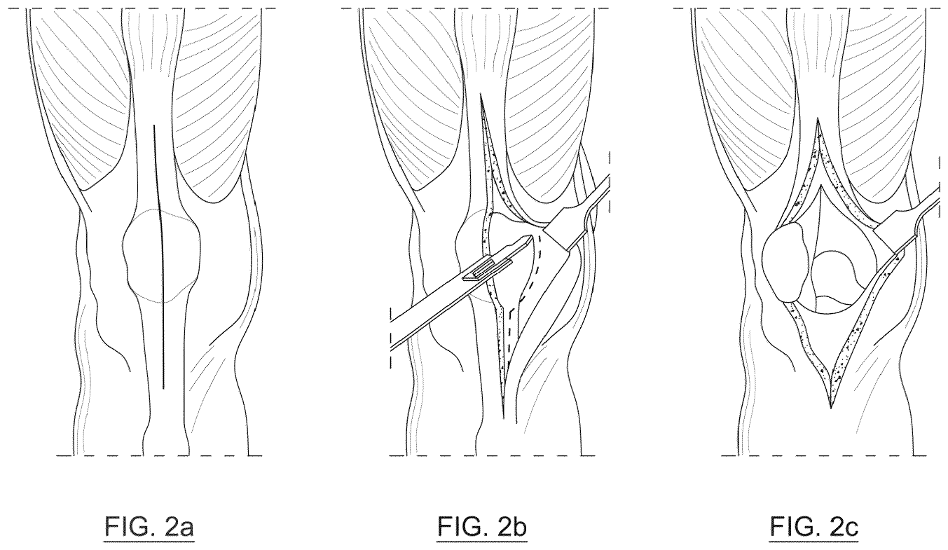

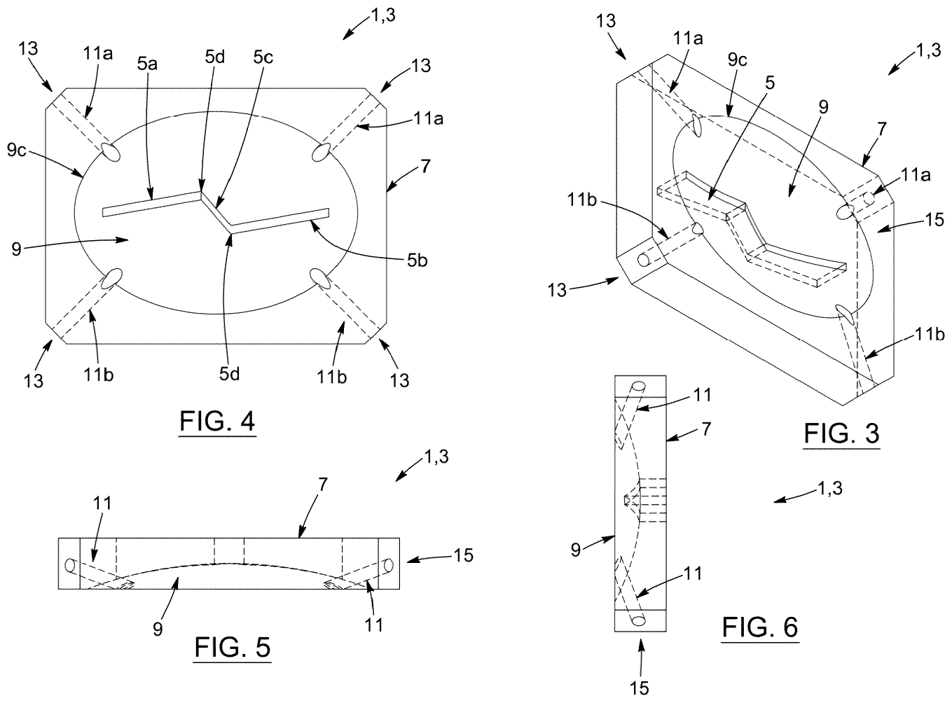

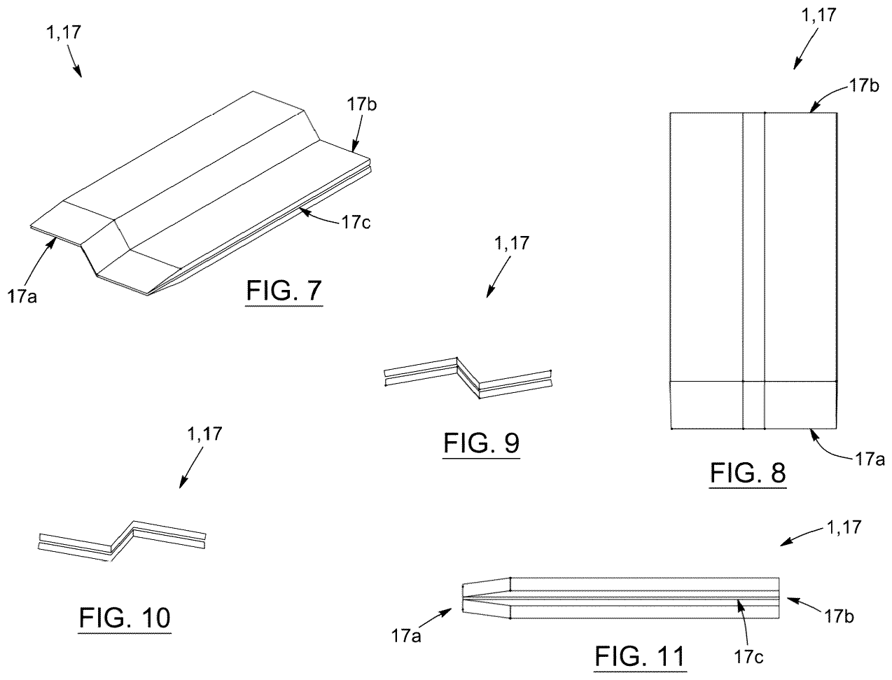

### DETAILED DESCRIPTION OF THE INVENTION

In the following description, like reference numerals refer to like components. The embodiments (geometries, dimensions, etc.) illustrated in the figures and the characteristics described in the application are preferential only, given as an indication only.

Furthermore, although the present invention is designed primarily for surgical intervention on a “knee” (ex. knee surgery, knee replacement, etc.), for example, the invention could be used with any other type of joints and/or for any other kind of application and/or useful purpose, as obvious to one skilled in the art. Therefore, expressions such as “intervention”, “surgery”, “knee”, “operation”, “replacement”, etc., as used herein, and/or any other reference and/or other equivalent expression or similar thereto are not to be construed as limiting the scope of the present invention and include any other objects/substitutes and/or other applications with which the present invention may be used and may be useful, as evident for a person skilled in the art.

Furthermore, although the preferred embodiments of the illustrated system comprise several components, all these components are not necessarily essential to the invention and consequently should not be taken in their restrictive sense, that is to say should not be considered to limit the scope of the present invention. It is to be understood therein, as obvious to a person skilled in the art, that other suitable components and geometries and/or other possible cooperations therebetween can be used for the system according to the present invention, such as that they will be described below, without departing from the scope of the invention.

Additionally, expressions such as “system”, “operating room”, “robot”, “controller”, “program”, “software”, “retrofit”, “control panel”, “kit”, “device”, “assembly”, “mechanism”, “product”, “device”, “operator”, etc., as well as any other equivalent expression and/or words composed of these, may be used interchangeably in the context of the present description, as obvious to a person skilled in the art. This also applies for other expressions which are mutually equivalent, such as, for example: a) “intervention”, “surgery”, “operation”, “repair”, “replacement”, “reconstruction”, “modification”, “inspection”, “arthroscopy”, “arthroplasty”, etc.; (b) “knee”, “joint”, “limb”, etc.; c) “section”, “cut”, “split”, “saw”, “separate”, “split”, etc.; d) “bone”, “patella”, etc.; (e) “screw”, “nail”, “anchor”, “clamp”, “connector”, “clip”, etc.; f) “controller”, “operator”, “robot”, “command”, “computer”, “circuit”, “hardware”, “software”, “software”, “mechanical component(s), electronic(s), electronic(s) and/or computer(s), etc.; as well as any other mutually equivalent expressions, related to the aforementioned expressions and/or to any other structural and/or functional aspects of the present invention, as also obvious to a person skilled in the art.

Additionally, in the context of this description, all elongated objects should be considered to implicitly have a “longitudinal axis” and/or a “centerline”, such as the longitudinal axis of a rod, for example, or the centerline of a duct, and that expressions such as “connected” and “connectable”, or “swiveled” and “swivelable”, may be interchanged and are mutually equivalent, in that the present invention also relates to a kit with corresponding components for assembling a resulting equipment and/or system fully assembled and operational to implement the present method and/or obtain any resulting and/or derived product.

In addition, certain components of this system and/or steps of this method (ex. assembly, operation, etc.) being described in this specification may be modified, simplified, omitted and/or interchanged, without departing from the scope of the present invention, depending on the particular applications for which the present system is designed and/or intended, and also depending on the results desired, as briefly described herein and as also obvious to a skilled person in the field.

In general, the present invention, as illustrated in the attached figures, relates to medical and/or surgical equipment allowing innovative knee surgery, for example, by reducing the disadvantages associated with other conventional ways of doing things, related systems and/or methods known in the prior art. The present invention also relates to a kit with corresponding components intended for assembly (ex. of the corresponding equipment, system and/or operating room, etc.), and/or to implement the resulting equipment, system and/or operating room(s), as well as methods of assembly, operation and/or use thereof.

As will be better understood by referring to the description which follows and the corresponding attached figures, the present equipment and/or corresponding operating system makes it possible to practice the new surgical approach proposed, either trans-patellar bone and tendon with longitudinal sectioning. This innovative approach could potentially remedy all the aforementioned drawbacks and thereby improve the results encountered with the conventional surgical approach to knee arthroplasty.

The reading of the following paragraphs, associated with the drawings, will make it possible to better understand how the advertised advantages are related to this technique.

### Overview of the New Surgical Approach Proposed Herein

Since this new approach does not affect the anatomical structures on the medial side of the patella, its functional impairment is avoided because no weakening has been created. For its part, the closure of the innovative approach that consists of passing through the patella, is done by rigid fixation of the two halves of the patella put in compression using implants, which can withstand very high tensions, and without undergoing deformation or weakening. In addition, the closure of the quadriceps tendon and the patellar tendon is done by bringing together using continuous threads of their respective fibers which have only been separated vertically and not cut. This results in very little bleeding, and almost zero tissue tension. This in turn would promote a significant reduction in pain during postoperative mobilization of the knee, and would allow greater range of motion to be achieved more quickly and easily.

This would represent a major advantage on the length of hospital stay, which in turn would significantly reduce the total costs generated for this type of operation.

Another major advantage of this new proposed approach is the quality and extent of surgical exposure offered in the absence of tension applied to the lateral and medial soft tissues during the procedure. Since the patella is split in two, it is no longer necessary to evert the patella which hinders much of the exposure of the knee and to exert local pressure due to its abnormal and dislocated position. It would also ensure a perfectly anatomical, symmetrical and watertight closure while respecting the integrity of all the tissues surrounding the patella. These advantages would lead to less pain, would make it possible to respond in a superior way to the extreme but not harmful demands of the early mobilization of the freshly operated knee and thus would guarantee an earlier and faster return to the activities of daily living (ADL).

Consequently, control of patellar alignment postoperatively compared to the standard surgical approach would not be altered. Nor would the distribution of tensile forces distributed over the soft tissues be affected by either the incision or its closure. In this new approach, muscle contraction of the vastus medialis oblique would exert its tensile force directly on the patella and not on the severed and then repaired tendon during closure in the conventional approach.

This new technique would obviously require new tools. A patella cutting guide is provided as well as a drill guide for screw or clamp, a patella separator, and a choice of implants for patella fixation. This would include an axial compression screw system, a double U-clamp system with stops and/or the use of a screw-leakage ring.

### Why Conventionnel Screws do not Constitute an Optimal Implant?

Although it would be possible to obtain an adequate assembly of the patella parts with the conventional screws, two disadvantages can hinder the desired rapid recovery towards a return to normal function of the knee:
1. The subcutaneous prominence of the screw head, if it is not sufficiently buried under the bone; and
2. The subcutaneous prominence from its tip to its exit from the bone, if longer than the width of the patella, could be a disturbing impingement problem.

An exact length of the screw respecting the exact width of the patella, including the cortical bone at the exit of the screw, is mandatory for obtaining an excellent grip in the bone. According to the opinion of the present Applicant/inventor, obtaining this degree of precision without compromising the quality of the fixing would be difficult to achieve with certainty each time this is necessary: too short, it would risk unbridling or loss of fixation, and too long, it would require a second operation after bone union to remove the fixation material, which would scrape under the skin.

In other words, an assembly performed with the conventional screw would exert an asymmetrical bone retention force on the two assembled parts. This would imply a significant risk of dismantling when this assembly is put under excessive tension, such as when bending beyond 90 degrees with a swollen knee, especially if combined with forced external rotation of the knee. The risk of unbridling could also arise following the cumulative effect of the deforming forces found in the repetition of movement during rehabilitation. As soon as the pain begins to decrease post-operatively, the repeated tensioning would add up to lead to the loosening of the screw before the start of bone healing, which normally occurs at around 6 weeks.

### CONCLUSION

The tensile forces that the peri-patellar tissues involved must undergo when rehabilitating a freshly operated knee for replacement are beyond what their repairs can withstand, without inevitably inducing an irreversible and permanent change at the very site of the defect. closing. The prioritization of early mobilization of the knee in the immediate postoperative period being crucial and essential, it becomes impossible to guarantee perfect healing at the same time with a return of the histological and anatomical integrity of the tissues involved and, secondarily, a return to normal function and alignment of the patella.

Conversely, if the principles of tissue healing (especially with regard to mechanical function) must be observed and applied according to the rules of the art, and if the rehabilitation of the operated knee respects its fundamental principles of healing, joint flexibility would be greatly affected. sacrificed, which would lead to a still greater proportion of poor functional results, by severe joint ankylosis of the knee.

It is for all these reasons that the proposed new surgical approach, either trans-patellar bone and tendon with longitudinal cut through the patella, could potentially remedy all the drawbacks encountered with the conventional surgical approach to knee arthroplasty; as cited above.

Namely that an interosseous healing is completed in a significantly less time, that it can also be completed with a significantly higher precision to restore the integrity of the anatomy, and achieve a resistance force far greater than that generated by early mobilization of the knee. This can be explained firstly by the richness of the vascular network around the patella from which its bone tissue benefits and secondly by the achievement of a highly rigid assembly force offered by the compression between the two bone fragments via the instrumentation. described and the different implants offered.

### REFERENCE NUMBERS

1. operating equipment (and corresponding tool(s) and/or accessory(ies))
3. sectioning guide (ex. “patellar cutting guide”)
5. groove (of the sectioning guide)
5a. first segment of the groove (ex. distal segment, rectilinear)
5b. second segment of the groove (ex. distal segment, rectilinear)
5c. other segment (ex. intermediate segment)
5d. transition segment (ex. between 2 neighboring segments)
7. main body (of the sectioning guide)
9. recess (of the sectioning guide)
9c. contour (of the sectioning guide)
11. passage (ex. side passage and/or other)
11a. first side passage (ex. lateral side, outer side, etc.)
11b. second side passage (ex. medial side, inner side, etc.)
13. corner (of sectioning guide)
15. thickness (ex. height-of sectioning guide)
17. sectioning tool (ex. patella separator)
17a. first edge (ex. cutting edge-of the sectioning tool)
17b. second edge (ex. non-cutting edge-of the sectioning tool)
17c. lateral groove (of the sectioning tool)
19. connection system (which can take on various embodiments)
19. connection system (ex. “U-shaped double clamps with stops”)
19. connection system (ex. “axial compression screw system”)
19. connection system (ex. “strapping and/or coiling”)
19. connection system (ex. “headless interfragmentary screw”)
19 connection system (ex. “screwable ring”)
21. attachment (ex. in the form of a “U”)
21a. first attachment
21b. second attachment
23. male part (of attachment)
25. female part (of attachment)
27. section (of the attachment)
29. first threaded head (ex. first “implant”)
29a. anchoring surface (of the 1^{st} threaded head)
29b. endpiece (of the 1^{st} threaded head)
29c. orifice (of the 1^{st} threaded head)
29d. peripheral contour(s) (of the 1^{st} threaded head)
29e. axial hole (of the 1^{st} threaded head)
31. second threaded head (ex. second “implant”)
31a. anchoring surface (of the 2^{nd} threaded head)
31b. endpiece (of the 2^{nd} threaded head)
31c. hole (of the 2^{nd} threaded head)
31d. peripheral contour (of the 2^{nd} threaded head)
31e. axial hole (of the 2^{nd} threaded head)
33. connecting rod
33a. first end (of the connecting rod)
33b. second end (of the connecting rod)
33c. main rod (of the connecting rod)
33d. head (of the connecting rod)
35. threading
37. component(s) (ex. miscellaneous, etc.)
39. first router bit (for 1^{st} threaded head)
41. second router bit (for 2^{nd} threaded head)
43. measuring tool (and/or “drill guide”)
43a. first main section (of the measuring tool)
43b. second main section (of the measuring tool)
43c. first complementary section (of the measurement tool)
43d. second complementary section (of the measuring tool)
43e. longitudinal axis (of the measuring tool)
45. measuring end (of the measuring tool)
45a. first measuring end (on first main section)
45b. second measuring end (on second main section)
47. measuring scale
49. reading line (for measurement scale)
51. tightening component
51a. first component of main tightening
51b. second component of main tightening
51c. first component of complementary tightening
51d. second component of complementary tightening
53. handle (ex. rounded and/or knurled-of tightening component)
53a. first handle (of 1^{st} component of main tightening)
53b. second handle (of 2^{nd} component of main tightening)
53c. first handle (of 1^{st} component of complementary tightening)
53c. second handle (of 2^{nd} component of complementary tightening)
55. wire (ex. strapping system)
57. hole (ex. for wire)
59. retention mechanism
61. interfragmentary screw
61a. first opposite piece (of interfragmentary screw)
61a. second opposite piece (of interfragmentary screw)
61c. longitudinal piece (of interfragmentary screw)
61d. distal cavity (of interfragmentary screw)
61l. longitudinal groove (of interfragmentary screw)
63. screwable ring
63a. internal peripheral part (of the screwable ring)
63b. external peripheral part (of the screwable ring)
63c. main body (of the screwable ring)
63d. distal portion (of the screwable ring)
63e. distal rim (of the screwable ring)
65. cavity (of the screwable ring)
67. rotating tool (ex. for screwable ring)
69. protruding element (of the rotating tool)
71. reinforcement part(s) (of the rotating tool)
73. common rod (of the rotating tool)
75. proximate manipulation portion (of the rotating tool)
75a. first opposite handle (of the manipulation portion)
75b. second opposite handle (of the manipulation portion)
77. grinding tool (and/or drilling, digging, plotting, etc.)

This operating equipment system (and various inventive aspects thereof) may take various forms and/or expressions, including one and/or more of the following components and features (and/or different(s) combination(s) and/or permutation(s) thereof, given as preferred and/or optional embodiment(s) only:
i.) An operating equipment (1) comprising a series of tools and/or accessories (in order to allow, in particular, a new surgical approach proposed hereby, either trans-patellar bone and tendon with longitudinal cutting, which could potentially remedy to all the drawbacks encountered with the conventional surgical approach to knee arthroplasty, and which would most likely be responsible for the poor results of this conventional intervention).
ii.) An operating equipment (1) according to any one of the preceding combinations, wherein the series of tools and/or accessories comprises a sectioning guide (3) (ex. sawing, splitting, cracking, separating, etc.) to cut into two sub-parts a bony/bone part (ex. patella) of the joint (ex. knee).
iii.) An operating equipment (1) according to any one of the preceding combinations, wherein the sectioning guide (3) comprises a groove (5) extending through a main body (7) of the sectioning guide (3) (ex. in a central part of the main body (7), for example), and defining a shape being intended to be used for severing the bone part (ex. patella) of the joint (ex. knee).
iv.) An operating equipment (1) according to any one of the preceding combinations, wherein the groove (5) of the sectioning guide (3) is segmented, and comprises at least two different segments (5a,5b).
v.) An operating equipment (1) according to any one of the preceding combinations, wherein the groove (5) of the sectioning guide (3) is segmented, and comprises at least three different segments (5a,5b,5c).
vi.) An operating equipment (1) according to any one of the preceding combinations, wherein each different segment (5a,5b,5c) from the groove (5) of the sectioning guide (3) is substantially rectilinear.
vii.) An operating equipment (1) according to any one of the preceding combinations, wherein each different rectilinear segment (5a, 5b, 5c) is connected to an adjacent rectilinear segment (5a,5b,5c) by via a transition segment (5d).
viii.) An operating equipment (1) according to any one of the preceding combinations, wherein the groove (5) of the sectioning guide (3) comprises first and second distal segments (5a,5b).
ix.) An operating equipment (1) according to any one of the preceding combinations, wherein the groove (5) of the sectioning guide (3) comprises at least one intermediate segment (5c).
x.) An operating equipment (1) according to any one of the preceding combinations, wherein the various segments (5a,5b,5c,5d) define the groove (5) of the sectioning guide (3).
xi.) An operating equipment (1) according to any one of the preceding combinations, wherein the groove (5) of the sectioning guide (3) defines a zigzag path.
xii.) An operating equipment (1) according to any one of the preceding combinations, wherein the sectioning guide (3) comprises a recess (9) being positioned, shaped and sized in order to receive a front portion of the part bone (ex. patella) of the joint (ex. knee).
xiii.) An operating equipment (1) according to any one of the preceding combinations, wherein the recess (9) has a substantially elliptical contour (9c).
xiv.) An operating equipment (1) according to any one of the preceding combinations, wherein the sectioning guide (3) comprises at least one passage (11) (ex. side passage and/or other) for receiving a mounting means (ex. a nail, a screw, an anchor, a clamp, etc.) for mounting the sectioning guide (3) selectively and temporarily on the frontal portion of the bone part (ex. patella) of the joint (ex. knee) being intended to be operated on surgically.
xv.) An operating equipment (1) according to any one of the preceding combinations, wherein the sectioning guide (3) has at least a first side passage (11a) in a first side of the sectioning guide (3) to connect the sectioning guide (3) selectively and temporarily on a first side (ex. lateral side, outer side, etc.) of the frontal portion of the bone part (ex. patella) of the joint (knee) intended to be operated on surgically, and wherein the sectioning guide (3) has at least a second side passage (11b) in a second side of the sectioning guide (3) for connecting the sectioning guide (3) selectively and temporarily on a second side (ex. medial side, interior side, etc.) of the frontal portion of the bone part (ex. patella) of the joint (ex. knee) intended to be operated on surgically.
xvi.) An operating equipment (1) according to any one of the preceding combinations, wherein the sectioning guide (3) comprises at least one pair of first side passages (11a) and at least one pair of second passages side (11b), in order to provide the sectioning guide (3) with a minimum of four side passages (11, 11a, 11b).
xvii.) An operating equipment (1) according to any one of the preceding combinations, wherein each passage (11, 11a, 11b) (ex. sideways and/or otherwise) is substantially cylindrical.
xviii.) An operating equipment (1) according to any one of the preceding combinations, wherein each passage (11, 11a, 11b) extends at an angle through the main body (7) of the sectioning guide (3).
xix.) An operating equipment (1) according to any one of the preceding combinations, wherein each passage (11, 11a, 11b) is provided with a first surface end of the sectioning guide (3) and a second end in an internal portion of the sectioning guide (3).
xx. An operating equipment (1) according to any one of the preceding combinations, wherein the first end of each passage (11, 11a, 11b) originates from a different corner (13) of the sectioning guide (3).
xxi.) An operating equipment (1) according to any one of the preceding combinations, wherein each corner (13) of the sectioning guide (3) is bevelled (ex. at an angle, with respect to lateral sides, etc.).
xxii.) An operating equipment (1) according to any one of the preceding combinations, wherein the second end of each passage (11, 11a, 11b) is directed (ex. oriented, converges, etc.) towards a central portion of the sectioning guide (3).
xxiii.) An operating equipment (1) according to any one of the preceding combinations, wherein the second end of each passage (11, 11a, 11b) opens into an internal portion of the sectioning guide (3).
xxiv.) An operating equipment (1) according to any one of the preceding combinations, wherein the second end of each passage (11, 11a, 11b) terminates in the recess (9) of the sectioning guide (3) being designed to receive a front portion of the bone part (ex. patella) of the joint (ex. knee) intended to be operated on surgically.
xxv.) An operating equipment (1) according to any one of the preceding combinations, wherein the sectioning guide (3) comprises a main body (7) being substantially rectangular.
xxvi.) An operating equipment (1) according to any one of the preceding combinations, wherein the main body (7) of the sectioning guide (3) has a given thickness (15) (ex. a given height, etc.).
xxvii.) An operating equipment (1) according to any one of the preceding combinations, wherein the recess (9) being designed to receive a frontal portion of the bone part (ex. patella) of the joint (ex. knee) intended to be operated on surgically is housed inside the given thickness (15) (ex. a given height, etc.) of the main body (7) of the sectioning guide (3).
xxviii.) An operating equipment (1) according to any one of the preceding combinations, wherein the series of tools and/or accessories comprises a sectioning tool (17) having a geometric profile complementary to the groove (5) of the sectioning guide (3), in order to allow insertion of said sectioning tool (17) through said groove (5) of the sectioning guide (3).
xxix.) An operating equipment (1) according to any one of the preceding combinations, wherein the sectioning tool (17) has a first edge (17a) (ex. a distal edge) which is sharp in order to allow cutting of the bone part (ex. patella) of the joint (ex. knee) into two corresponding sub-parts.
xxx.) An operating equipment (1) according to any one of the preceding combinations, wherein the sectioning tool (17) has a second edge (17b) (ex. a near edge) which is not sharp in order to receive a tool (ex. a hammer, etc.) for striking the sectioning tool (17) and thereby allowing the first edge (17a) (ex. a distal edge) to be cut to section the bone part (ex. patella) of the joint (ex. knee) into two corresponding sub-parts.
xxxi.) An operating equipment (1) according to any one of the preceding combinations, wherein the sectioning tool (17) comprises at least one lateral groove (17c).
xxxii.) An operating equipment (1) according to any one of the preceding combinations, wherein the sectioning tool (17) has a pair of lateral grooves (17c).
xxxiii.) An operating equipment (1) according to any one of the preceding combinations, wherein the series of tools and/or accessories comprises a connection system (19) for reconnecting the bone part (ex. patella) again and together of the joint (ex. knee) having been sectioned into two sub-parts with the guide and/or the sectioning tool (17).
xxxiv.) An operating equipment (1) according to any one of the preceding combinations, wherein the connection system (19) comprises at least one attachment (21) being insertable into a corresponding hole having been defined (ex. perforated, etc.) in the two corresponding sub-parts of the bone part (ex. patella) of the joint (ex. knee) having been severed, the attachment (21) being able to be adjusted and tightened in order to bring the two sub-parts of the bone part (ex. patella) of the joint (ex. knee) together after surgery.
xxxv. An operating equipment (1) according to any one of the preceding combinations, wherein said at least one attachment (21) comprises first and second attachments (21a,21b), at least one of these attachments (21a,21b) comprising at least one male part (23) intended to cooperate with at least one corresponding female part (25) of the other attachment (21b,21a), to allow corresponding interconnection and self-locking between said attachments (21a,21b).
xxxvi.) An operating equipment (1) according to any one of the preceding combinations, wherein the first and second attachments (21a,21b) each comprise a series of male and female parts (23,25) intended to cooperate with the corresponding female and male parts (25,23) of the other attachment (21b,21a).
xxxvii.) An operating equipment (1) according to any one of the preceding combinations, wherein each attachment (21a,21b) is substantially “U” shaped, and comprises two sections (27) each being provided with a series of male and female parts (23,25) intended to cooperate with the corresponding female and male parts (25,23) of the other attachment (21b,21b).
xxxviii.) An operating equipment (1) according to any one of the preceding combinations, wherein each section (27) of each attachment (21a,21b) is provided with a series of male and female parts (23,25) being defined by alternating highs and lows.
xxxix.) An operating equipment (1) according to any one of the preceding combinations, wherein each section (27) of each attachment (21a,21b) has a cross-section which decreases towards a distal end of said each section.
xl.) An operating equipment (1) according to any one of the preceding combinations, wherein the connection system (19) comprises a first threaded head (29) (ex. first “implant”) being insertable into a first sub-part of the two sub-parts of the bone part (ex. patella) of the joint (ex. knee) having been sectioned with the guide and/or the sectioning tool (17), a second threaded head (31) (ex. second “implant”) being insertable into a second sub-part of the two sub-parts of the bone part (ex. patella) of the joint (ex. knee) having been severed, and a connection rod (33) cooperating with the two threaded heads (29,31), the connection system (19) being able to be adjusted and tightened by means of a rotation of the connection rod (33) in order to bring the two sub-parts of the bone part (ex. patella) of the joint (ex. knee) back together after surgery.
xli.) An operating equipment (1) according to any one of the preceding combinations, wherein the first threaded head (29) comprises an anchoring surface (29a) being provided with threading (35) for anchoring (ex inserting, penetrating, aiming, etc.) this first threaded head (29) in the first sub-part of the two sub-parts of the bone part (ex. patella) of the joint (ex. knee).
xlii.) An operating equipment (1) according to any one of the preceding combinations, wherein the anchoring surface (29a) of the first threaded head (29) is tapered (ex. at an angle, etc.).
xliii.) An operating equipment (1) according to any one of the preceding combinations, wherein the first threaded head (29) is provided with a endpiece (29b) comprising at least one orifice (29c) for receiving a tool for rotating and/or affixing (ex. anchoring, engaging, inserting, etc.) and holding in place the first threaded head (29) in the first sub-part of the two sub-parts of the bone part (ex. patella) of the joint (ex. knee) (ex. before, and/or during an adjustment and/or tightening of the connection system (19)).
xliv.) An operating equipment (1) according to any one of the preceding combinations, wherein the endpiece (29b) of the first threaded head (29) has n corresponding orifices (29c), n being an integer being greater than 1 (ex. n=2, 3, 4, 5, etc.).
xlv.) An operating equipment (1) according to any one of the preceding combinations, wherein the n orifices (29c) of the endpiece (29b) of the first threaded head (29) are identical with respect to each other.
xlvi.) An operating equipment (1) according to any one of the preceding combinations, wherein the n orifices (29c) of the endpiece (29b) of the first threaded head (29) are equidistant from one to the other.
xlvii.) An operating equipment (1) according to any one of the preceding combinations, wherein the end piece (29b) of the first threaded head (29) has 3 corresponding orifices (29c).
xlviii.) An operating equipment (1) according to any one of the preceding combinations, wherein the endpiece (29b) of the first threaded head (29) has rounded peripheral contours (29d).
xlix.) An operating equipment (1) according to any one of the preceding combinations, wherein the endpiece (29b) of the first threaded head (29) has an axial hole (29e) (ex. longitudinal, etc.) being configured to receive a first end (33a) of the connecting rod (33).
l.) An operating equipment (1) according to any one of the preceding combinations, wherein the axial hole (29e) (ex. longitudinal, etc.) of the first threaded head (29) is provided with threading (35) configured to receive a corresponding external threading (35) of the first end (33a) of the connecting rod (33).
li.) An operating equipment (1) according to any one of the preceding combinations, wherein the second threaded head (31) comprises an anchoring surface (31a) being provided with threading (35) for anchoring (ex. insert, penetrate, engage, etc.) this second threaded head (31) in the second sub-part of the two sub-parts of the bone part (ex. patella) of the joint (ex. knee).
lii.) An operating equipment (1) according to any one of the preceding combinations, wherein the anchoring surface (31a) of the second threaded head (31) is curved.
liii.) An operating equipment (1) according to any one of the preceding combinations, wherein the second threaded head (31) is provided with a endpiece (31b) comprising at least one orifice (31c) for receiving a tool for rotating and/or affixing (ex. anchoring, engaging, inserting, etc.) and holding in place the second threaded head (31) in the second sub-part of the two sub-parts of the bone part (ex. patella) of the joint (ex. knee) (ex. before, and/or during an adjustment and/or tightening of the connection system (19)).
liv.) An operating equipment (1) according to any one of the preceding combinations, wherein the endpiece (31b) of the second threaded head (31) has n corresponding orifices (31c), n being a number integer being greater than 1 (ex. n=2, 3, 4, 5, etc.).
lv.) An operating equipment (1) according to any one of the preceding combinations, wherein the n orifices (31c) of the endpiece (31b) of the second threaded head (31) are identical with respect to each other.
lvi.) An operating equipment (1) according to any one of the preceding combinations, wherein the n orifices (31c) of the endpiece (31b) of the second threaded head (31) are equidistant from one to the other.
lvii.) An operating equipment (1) according to any one of the preceding combinations, wherein the endpiece (31b) of the second threaded head (31) has 4 corresponding orifices (29c).
lviii.) An operating equipment (1) according to any one of the preceding combinations, wherein the endpiece (31b) of the second threaded head (31) has rounded peripheral contours (31d).
lix.) An operating equipment (1) according to any one of the preceding combinations, wherein the endpiece (31b) of the second threaded head (31) comprises an axial hole (31e) (ex. longitudinal, etc.) being configured to receive a second end (33b) of the connecting rod (33).
lx.) An operating equipment (1) according to any one of the preceding combinations, wherein the axial hole (31e) (ex. longitudinal, etc.) of the second threaded head (31) is configured to leave a passage of the first end (33a) of the connecting rod (33), beforehand, so that this first end (33a) of the connecting rod (33) is connected (ex. mounted, inserted, screwed, etc.) on the first threaded head (29).
lxi.) An operating equipment (1) according to any one of the preceding combinations, wherein the connecting rod (33) comprises a main rod (33c), a first end (33a) provided with an external threading (35) being complementary to a corresponding internal threading (35) of the axial hole (29e) (ex. longitudinal) of the first threaded head (29), and a second end (33b) provided with a corresponding head (33d) provided with a cavity (ex. socket) to receive a corresponding turning tool (ex. screwdriver, etc.).
lxii.) An operating equipment (1) according to any one of the preceding combinations, wherein the series of tools and/or accessories comprises a corresponding first router bit (39) (ex. drill bit, etc.) having a geometric profile being substantially complementary to that of the main body of the first threaded head (29), the first router bit (39) being used to drill a corresponding hole in the first sub-part of the two sub-parts of the bone part (ex. patella) of the joint (ex. knee) intended to receive the first threaded head (29).
lxiii.) An operating equipment (1) according to any one of the preceding combinations, wherein the series of tools and/or accessories comprises a second router bit (41) (ex. drill bit, etc.) corresponding having a geometric profile being substantially complementary to that of the main body of the second threaded head (31), the second router bit (41) being used to drill a corresponding hole in the second sub-part of the two sub-parts the bone part (ex. patella) of the joint (ex. knee) intended to receive the second threaded head (31).
lxiv.) An operating equipment (1) according to any one of the preceding combinations, wherein the series of tools and/or accessories comprises a measuring tool (43) (and/or “drill guide”) to measure a distance relative to the corresponding hole to be defined (ex. perforate, etc.) in the two corresponding sub-parts of the bone part (ex. patella) of the joint (ex. knee) to be cut, in order to be able to proceed with the surgery and then be able to better bring the two sub-parts of the bone part (ex. patella) of the joint (ex. knee) back together after the surgery.
lxv.) An operating equipment (1) according to any one of the preceding combinations, wherein the measuring tool (43) comprises two main sections (43a,43b) being movable (ex. sliding, etc.) one relative to the other.
lxvi.) An operating equipment (1) according to any one of the preceding combinations, wherein the measuring tool (43) has two measuring ends (45a,45b) being movable (ex. sliding, etc.) relative to each other through relative movement between the two main sections (43a,43b) of the measuring tool (43).
lxvii.) An operating equipment (1) according to any one of the preceding combinations, wherein at least one main section (43a,43b) of the measuring tool (43) comprises a measuring scale (47) and a corresponding reading line (49), in order to be able to measure a distance between the two measuring ends (45a,45b) of the measuring tool (43).
lxviii.) An operating equipment (1) according to any one of the preceding combinations, wherein each main section (43a,43b) (ex. the two main sections) of the measuring tool (43) comprises a scale (47) and a corresponding reading line (49), in order to be able to measure a distance between the two measuring ends (45a,45b) of the measuring tool (43).
lxix.) An operating equipment (1) according to any one of the preceding combinations, wherein the measuring tool (43) comprises at least one main tightening component (51) (ex. blocking, etc.) in order to hold in place in a fixedly manner, the two main sections (43a,43b), and therefore, the two corresponding measuring ends (45a,45b), of the measuring tool (43), one with respect to the other.
lxx.) An operating equipment (1) according to any one of the preceding combinations, wherein said at least one main tightening component (51) (ex. locking, etc.) comprises a handle (53,53a,53b).
lxxi.) An operating equipment (1) according to any one of the preceding combinations, wherein the handle (53,53a,53b) of said at least one tightening component (51) (ex. locking, etc.) main is rounded.
lxxii.) An operating equipment (1) according to any one of the preceding combinations, wherein the handle (53,53a,53b) of said at least one main tightening component (51) (ex. locking, etc.) has a knurled peripheral surface to allow an increased manual grip by a user of the measuring tool (43).
lxxiii.) An operating equipment (1) according to any one of the preceding combinations, wherein said at least one main tightening component (51) (ex. locking, etc.) comprises two main tightening components (51a,51b) (ex. blocking, etc.), one for each main section (43a,43b) of the measuring tool (43), in order to hold the two main sections (43a,43b) in a fixedly manner, and therefore, the two corresponding measuring ends (45a,45b) of the measuring tool (43), relative to each other.
lxxiv.) An operating equipment (1) according to any one of the preceding combinations, wherein the measuring tool (43) comprises at least one complementary section (43c, 43d) being movable (ex. sliding, etc.) with respect to a main body of the measuring tool (43), a measuring end (45,45a,45b) of the measuring tool (43) being mounted on said complementary section (43c,43d).
lxxv.) An operating equipment (1) according to any one of the preceding combinations, wherein the measuring tool (43) comprises two complementary sections (43c,43d) each being movable (ex. slidable, etc.) relative to a main body of the measuring tool (43), a measuring end (45,45a,45b) of the measuring tool (43) being mounted on each of said complementary sections (43c,43d).
lxxvi.) An operating equipment (1) according to any one of the preceding combinations, wherein at least one complementary section (43c,43d) of the measuring tool (43) comprises a measuring scale (47) and a corresponding reading line (49), in order to be able to measure a distance between a longitudinal axis (43e) of the measuring tool (43) and a corresponding measuring end (45,45a,45b).
lxxvii.) An operating equipment (1) according to any one of the preceding combinations, wherein each complementary section (43c,43d) (ex. the two complementary sections) of the measuring tool (43) comprises a scale (47) and a corresponding reading line (49), in order to be able to measure a distance between a longitudinal axis (43e) of the measuring tool (43) and a corresponding measuring end (45,45a,45b) associated with a given complementary section (43c,43d).
lxxviii.) An operating equipment (1) according to any one of the preceding combinations, wherein the measuring tool (43) comprises at least one complementary tightening component (ex. blocking, etc.) (51c,51d) in order to hold in place, at least one complementary section (43c,43d) and a corresponding measuring end (45,45a,45b), in a fixedly manner with respect to a main body of the measuring tool (43).
lxxix.) An operating equipment (1) according to any one of the preceding combinations, wherein said at least one complementary tightening component (ex. blocking, etc.) (51c,51d) comprises a handle (53,53c,53d).
lxxx.) An operating equipment (1) according to any one of the preceding combinations, wherein the handle (53,53c,53d) of said at least one complementary tightening component (ex. locking, etc.) (51c,51d) is rounded.
lxxxi.) An operating equipment (1) according to any one of the preceding combinations, wherein the handle (53,53c,53d) of said at least one complementary tightening component (ex. blocking, etc.) (51c,51d) has a knurled peripheral surface to allow an increased manual grip by a user of the measuring tool (43).
lxxxii.) An operating equipment (1) according to any one of the preceding combinations, wherein said at least one complementary tightening component (ex. blocking, etc.) (51c,51d) comprises two tightening components (ex. blocking, etc.) (51c,51d), one for each complementary section (43c,43d) of the measuring tool (43), in order to hold in place, each complementary section (43c,43d) and a corresponding measuring end (45,45a,45b), fixedly relative to a main body of the measuring tool (43).
lxxxiii.) An operating equipment (1) according to any one of the preceding combinations, wherein the connection system (19) comprises a strapping system (ex. winding, etc.).
lxxxiv.) An operating equipment (1) according to any one of the preceding combinations, wherein the strapping system is selectively tensioned by means of a tensioner.
lxxxv.) An operating equipment (1) according to any one of the preceding combinations, wherein the strapping system comprises at least one wire (55).
lxxxvi.) An operating equipment (1) according to any one of the preceding combinations, wherein said at least one wire (55) of the strapping system comprises at least one wire (55) of high tension.
lxxxvii.) An operating equipment (1) according to any one of the preceding combinations, wherein said at least one wire (55) of the strapping system comprises at least one metallic wire (55) (ex. filament, etc.).
lxxxviii.) An operating equipment (1) according to any one of the preceding combinations, wherein said at least one wire (55) of the strapping system comprises a plurality of metallic filaments being braided together.
lxxxix.) An operating equipment (1) according to any one of the preceding combinations, wherein said at least one wire (55) of the strapping system is insertable into at least one corresponding hole (57) of the patella.
xc.) An operating equipment (1) according to any one of the preceding combinations, wherein said at least one wire (55) of the strapping system is adapted to be fixed in place by a retention mechanism (59) (ex. a knot being closed and hidden behind and/or in a recess (9), for example).
xci.) An operating equipment (1) according to any one of the preceding combinations, wherein the connection system (19) comprises a headless (ex. without a head) interfragmentary screw system (61), having a longitudinal piece (61c), and first and second opposite pieces (61a,61b) with outer threaded surfaces, these first and second pieces (61a,61b) being mountable and movable along said longitudinal piece (61c).
xcii.) An operating equipment (1) according to any one of the preceding combinations, wherein geometrical arrangements (ex. thread type, thread pitch, thread shape, etc.) of the first and second opposed pieces (61a,61b) are different from each other.
xciii.) An operating equipment (1) according to any one of the preceding combinations, wherein each opposite piece (61a,61b) has a distal cavity (61d) being positioned, shaped and sized to receive a tool corresponding intended to screw (ex. tighten, etc.) and/or to unscrew the first and second opposite pieces (61a,61b) in movement with respect to each other along the longitudinal piece (61c) of the interfragmentary screw system (61) without a head.
xciv.) An operating equipment (1) according to any one of the preceding combinations, wherein the longitudinal piece (61c) of the headless interfragmentary screw system (61) comprises at least one longitudinal groove (61l).
xcv.) An operating equipment (1) according to any one of the preceding combinations, wherein the longitudinal piece (61c) of the headless interfragmentary screw system (61) comprises at least one peripheral longitudinal groove (611) and/or at least one axial longitudinal groove (611).
xcvi.) An operating equipment (1) according to any one of the preceding combinations, wherein the connection system (19) comprises a screwable ring (63).
xcvii.) An operating equipment (1) according to any one of the preceding combinations, wherein the screwable ring (63) has an inner peripheral part (63a) and an outer peripheral part (63b).
xcviii.) An operating equipment (1) according to any one of the preceding combinations, wherein at least a peripheral part of the screwable ring (63) is provided with a threaded surface.
xcix.) An operating equipment (1) according to any one of the preceding combinations, wherein the two peripheral parts of the screwable ring (63) (i.e. inner and outer parts) are provided with respective threaded surfaces.
c.) An operating equipment (1) according to any one of the preceding combinations, wherein the screwable ring (63) comprises a main body (63c) being substantially circular (ex. in order to allow a corresponding screwing and unscrewing of the ring, etc.).
ci.) An operating equipment (1) according to any one of the preceding combinations, wherein the main body (63c) of the screwable ring (63) comprises a cross-section being profiled (ex. angled, converging down, etc.).
cii.) An operating equipment (1) according to any one of the preceding combinations, wherein the cross-section of the main body (63c) of the screwable ring (63) has a distal portion (63d).
ciii.) An operating equipment (1) according to any one of the preceding combinations, wherein the distal portion (63d) of the cross-section of the main body (63c) of the screwable ring (63) is substantially pointy (ex. triangular shape, etc.).
civ.) An operating equipment (1) according to any one of the preceding combinations, wherein the distal portion (63d) of the sectional section of the main body (63c) of the screwable ring (63) has a distal rim (63e).
cv.) An operating equipment (1) according to any one of the preceding combinations, wherein the distal edge (63e) of the cross-section of the main body (63c) of the screwable ring (63) is substantially sharp (ex. penetrating, etc.).
cvi.) An operating equipment (1) according to any one of the preceding combinations, wherein an upper part of the main body (63c) of the screwable ring (63) is provided with at least one cavity (65) corresponding to receive a corresponding rotating tool (67) (ex. for screwing/unscrewing, etc.).
cvii.) An operating equipment (1) according to any one of the preceding combinations, wherein the upper part of the main body (63c) of the screwable ring (63) is provided with 2n corresponding cavities (65) for receiving the corresponding rotating tool (67), n being an integer being equal to and/or greater than 1 (ex. n=1, 2, 3, 4, 5, etc.).
cviii.) An operating equipment (1) according to any one of the preceding combinations, wherein each corresponding cavity (65) of the screwable ring (63) is adapted to receive a corresponding protruding element (69) of the corresponding rotating tool (67).
cix.) An operating equipment (1) according to any one of the preceding combinations, wherein each corresponding cavity (65) of the screwable ring (63) is arranged around the main body (63c) of the screwable ring (63).
cx.) An operating equipment (1) according to any one of the preceding combinations, wherein each corresponding cavity (65) of the screwable ring (63) is arranged equidistant from each other around the main body (63c) of the screwable ring (63).
cxi.) An operating equipment (1) according to any one of the preceding combinations, wherein the upper part of the main body (63c) of the screwable ring (63) is provided with at least two corresponding cavities (65) (ex. n=1, and therefore, 2n=2) to receive protruding elements (69) of the corresponding rotating tool (67).
cxii.) An operating equipment (1) according to any one of the preceding combinations, wherein the upper part of the main body (63c) of the screwable ring (63) is provided with at least four corresponding cavities (65) (ex. n=2, and therefore, 2n=4) to receive protruding elements (69) of the corresponding rotating tool (67).
cxiii.) An operating equipment (1) according to any one of the preceding combinations, wherein the upper part of the main body (63c) of the screwable ring (63) is provided with at least six corresponding cavities (65) (ex. n=3, and therefore, 2n=3) to receive protruding elements (69) of the corresponding rotating tool (67).
cxiv.) An operating equipment (1) according to any one of the preceding combinations, wherein the upper part of the main body (63c) of the screwable ring (63) is provided with at least eight corresponding cavities (65) (ex. n=4, and therefore, 2n=8) to receive protruding elements (69) of the corresponding rotating tool (67).
cxv.) An operating equipment (1) according to any one of the preceding combinations, wherein the corresponding rotating tool (67) (ex. for screwing/unscrewing, etc.) comprises a number of protruding elements (69) being complementary (and/or compatible, etc.) with the number of corresponding cavities (65) provided on the screwable ring (63).
cxvi.) An operating equipment (1) according to any one of the preceding combinations, wherein each protruding element (69) of the rotating tool (67) (ex. for screwing/unscrewing, etc.) is complementary (ex. in shape and/or in size) to at least one cavity (65) corresponding to the screwable ring (63).
cxvii.) An operating equipment (1) according to any one of the preceding combinations, wherein each protruding element (69) of the rotating tool (67) (ex. for screwing/unscrewing, etc.) is complementary (ex. in shape and/or in size) to each corresponding cavity (65) of the screwable ring (63).
cxviii.) An operating equipment (1) according to any one of the preceding combinations, wherein each corresponding cavity (65) of the screwable ring (63) is substantially cylindrical, and therefore, wherein each protruding element (69) of the corresponding rotating tool (67) (ex. for screwing/unscrewing, etc.) is also substantially cylindrical.
cxix.) An operating equipment (1) according to any one of the preceding combinations, wherein each protruding element (69) of the rotating tool (67) (ex. for screwing/unscrewing, etc.) is arranged on a distal part of the rotating tool (67) (ex. for screwing/unscrewing, etc.).
cxx.) An operating equipment (1) according to any one of the preceding combinations, wherein the distal part of the rotating tool (67) (ex. for screwing/unscrewing, etc.) is substantially circular and complementary to the upper part of the screwable ring (63).
cxxi.) An operating equipment (1) according to any one of the preceding combinations, wherein the distal part of the rotating tool (67) is provided with reinforcement parts (ex. bars and/or the like) (71) converging into a common rod (73).
cxxii.) An operating equipment (1) according to any one of the preceding combinations, wherein the rotating tool (67) (ex. for screwing/unscrewing, etc.) is provided with a proximate manipulation portion (75) (ex. manual handling and/or other).
cxxiii.) An operating equipment (1) according to any one of the preceding combinations, wherein the proximate manipulation portion (75) (ex. manual handling and/or other) is provided on a common rod (73) of the rotating tool (67) (ex. for screwing/unscrewing, etc.).
cxxiv.) An operating equipment (1) according to any one of the preceding combinations, wherein the proximate manipulation portion (75) comprises a pair of opposite handles (75a, 75b) (ex. in order to allow a user of the tool to transmit a greater torque to the tool and, consequently, to the screwable ring (63), etc.).
cxxv. An operating equipment (1) according to any one of the preceding combinations, wherein the operating equipment (1) incudes a tool (ex. a bit, a bell, a saw, etc.) for grinding (77) (and/or drilling, etc.) having a substantially complementary geometric profile (ex. substantially similar and/or smaller, etc.) to that of the main body (63c) of the screwable ring (63), to allow to hollow out a corresponding circular depression beforehand in the patella, being intended to receive said screwable ring (63).
cxxvi.) An operating system provided with an operating equipment (1) according to any one of the preceding combinations.

cxxvii.) A use of a tool and/or accessory (and/or part and/or component thereof) of the operating equipment (1), according to any one of the preceding combinations.
cxxviii.) A method of operation (ex. surgery) using an aspect of the operating equipment (1) according to any one of the preceding combinations.

Of course, several modifications and/or other alternatives and/or other technical/mechanical equivalents could be made to this system and/or to the components and/or to the corresponding steps thereof, without departing from the present invention, as obvious to a person skilled in the art.

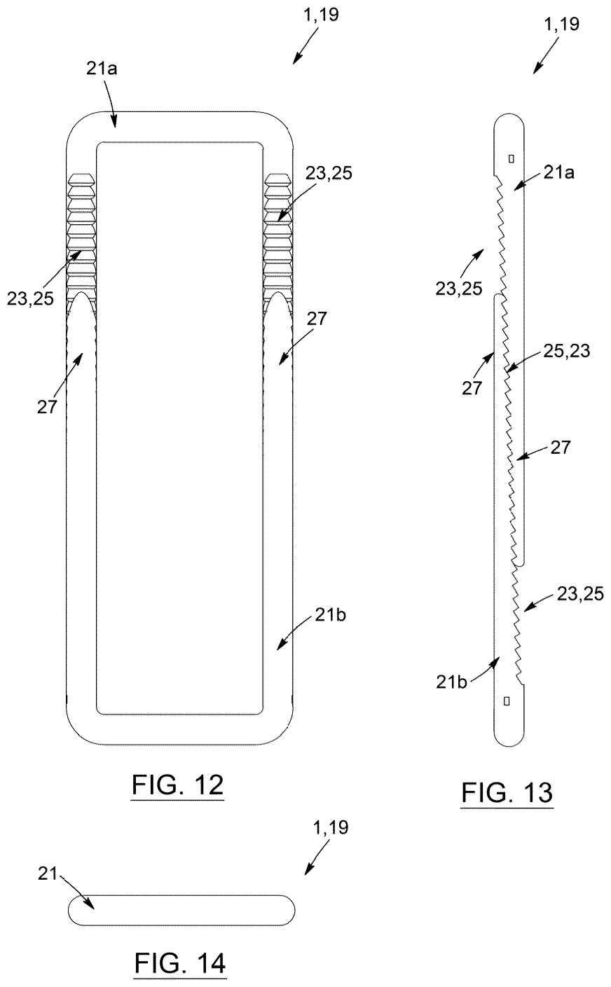

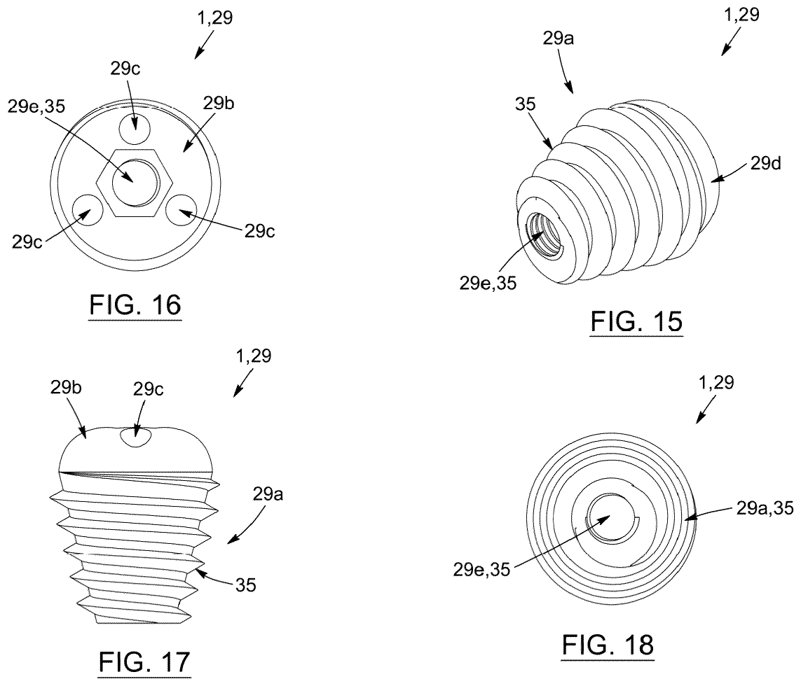

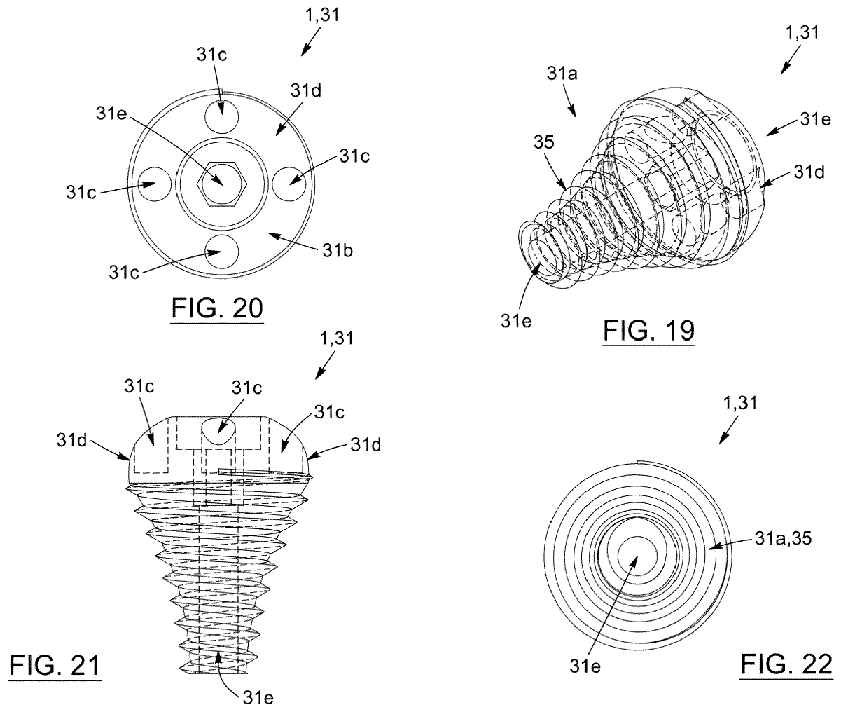

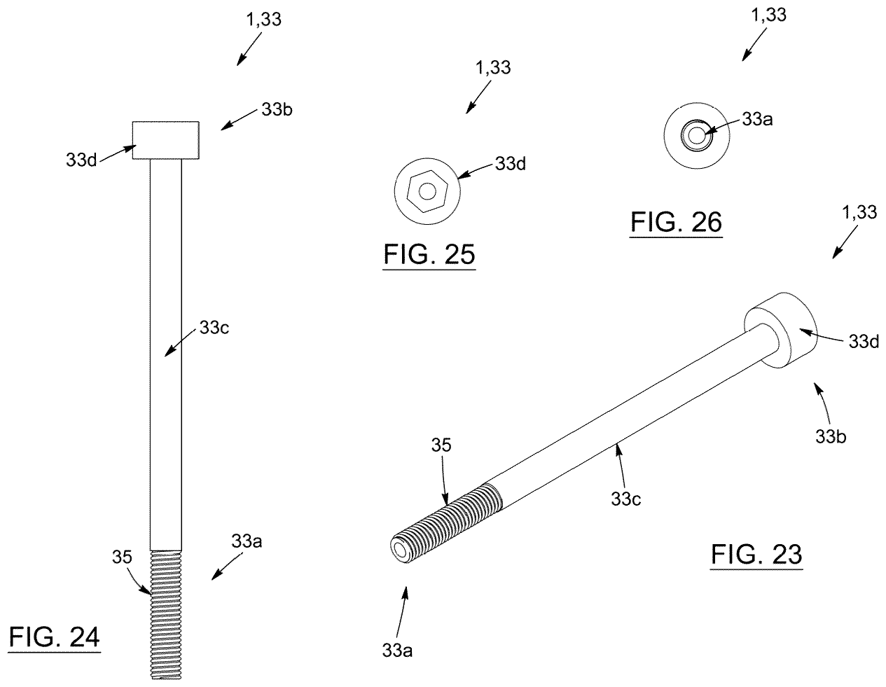

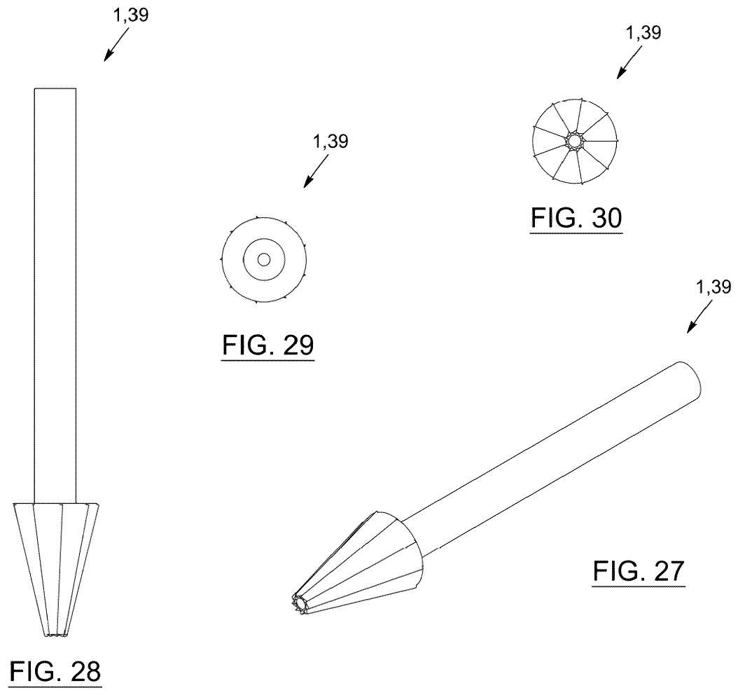

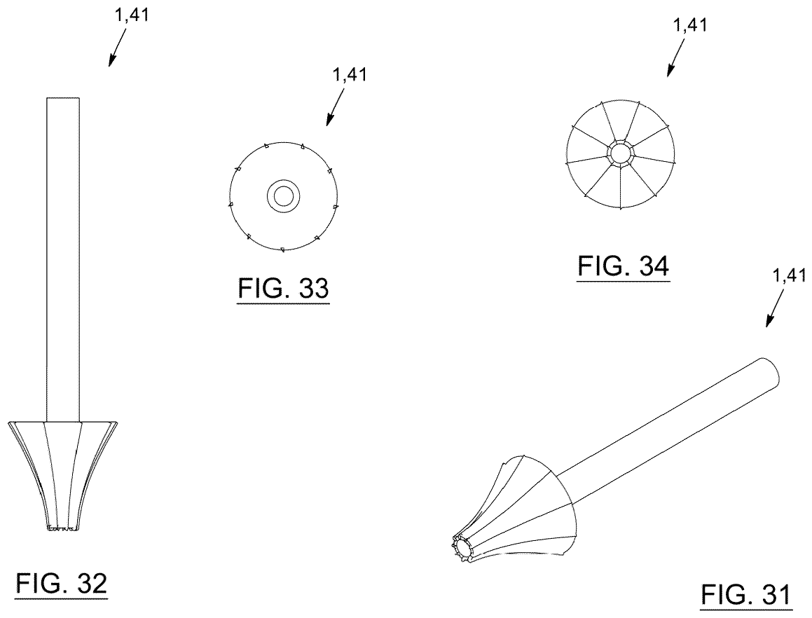

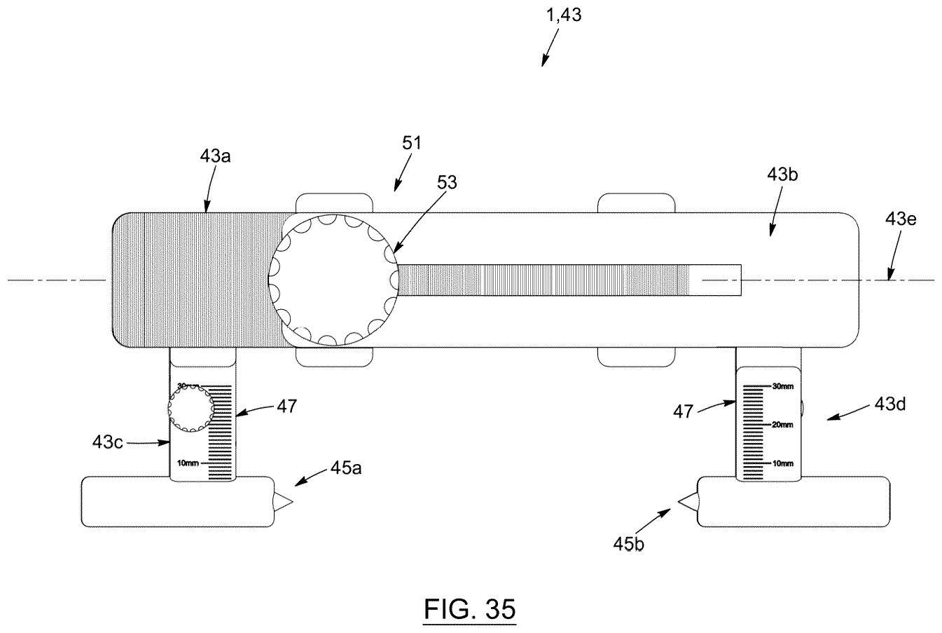

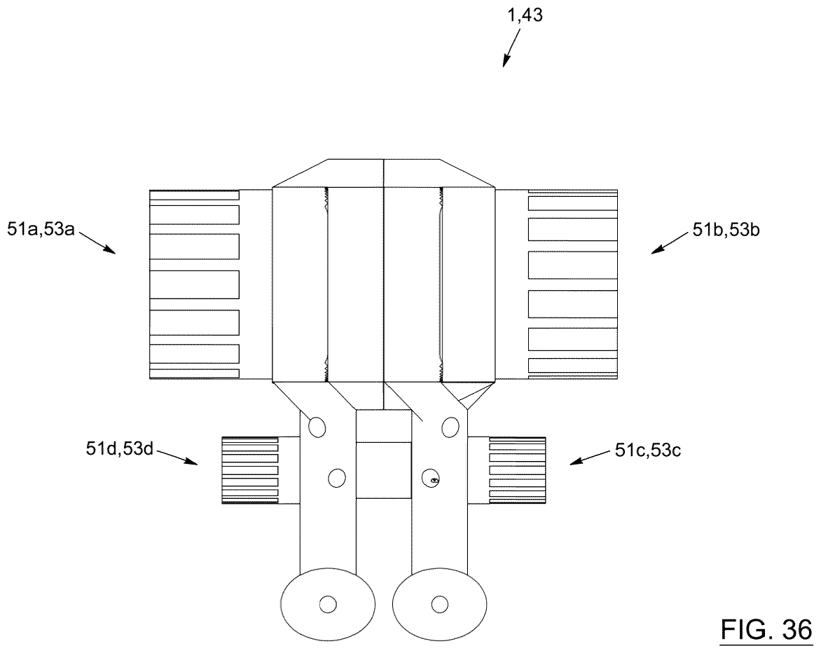

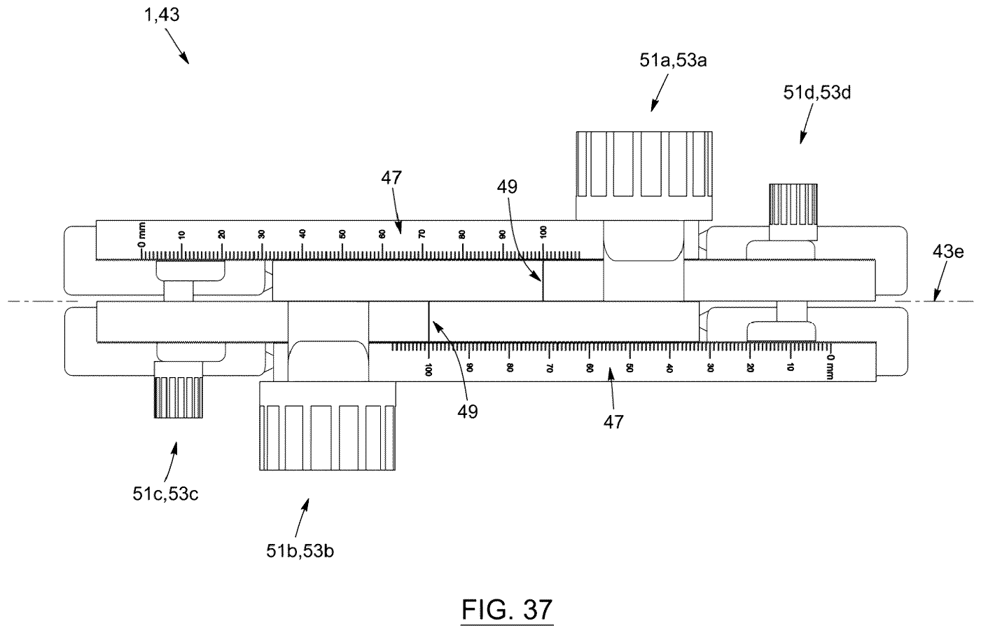

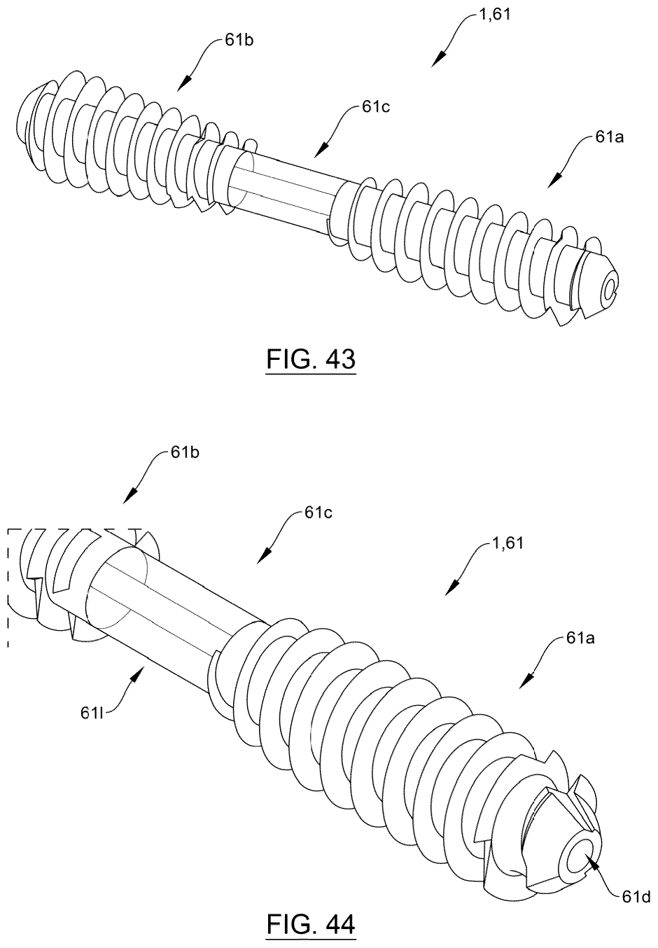

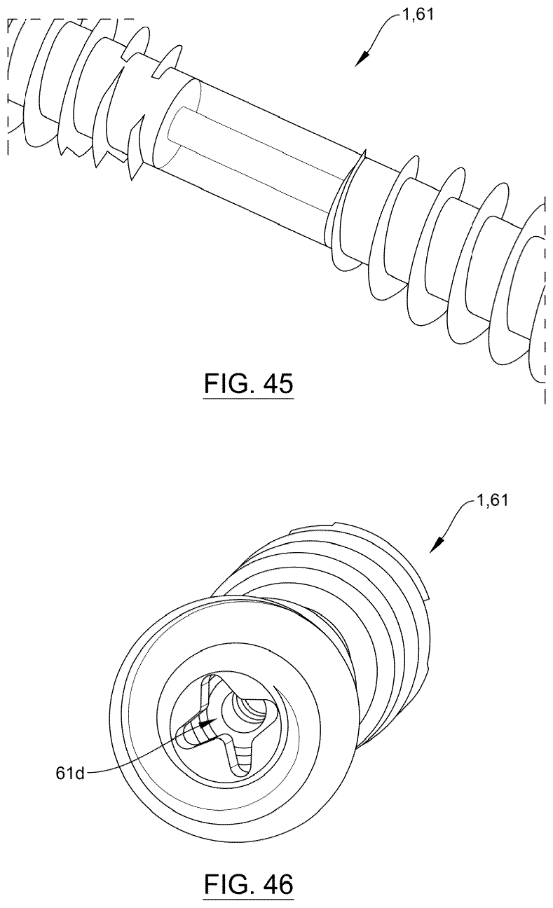

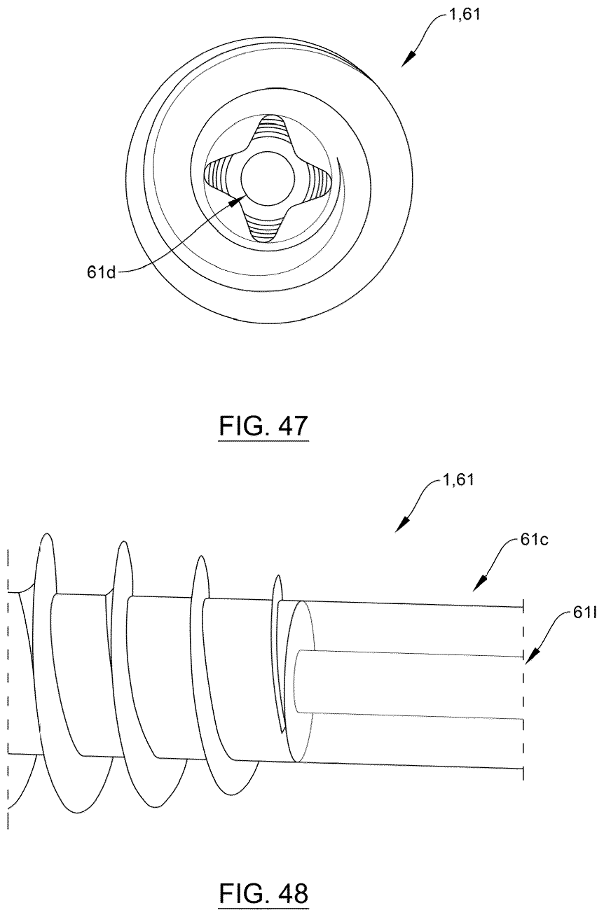

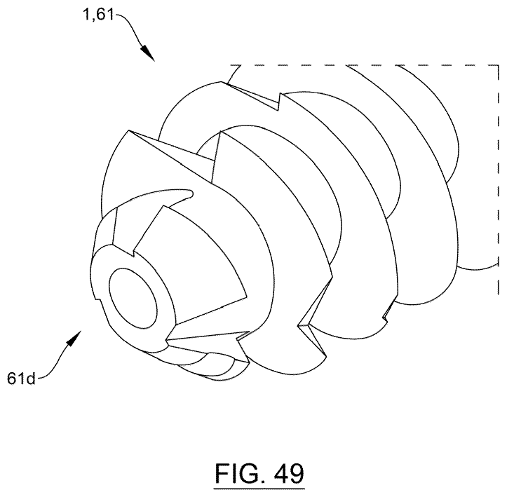

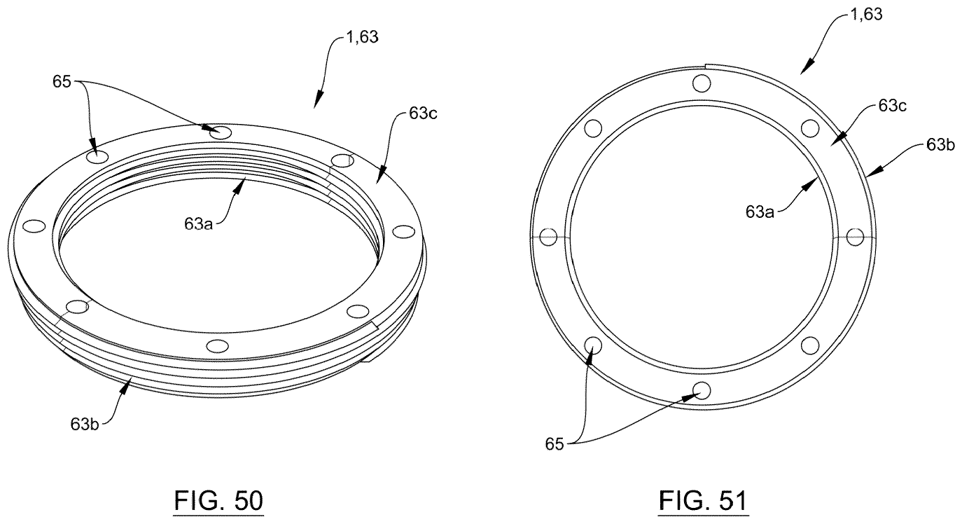

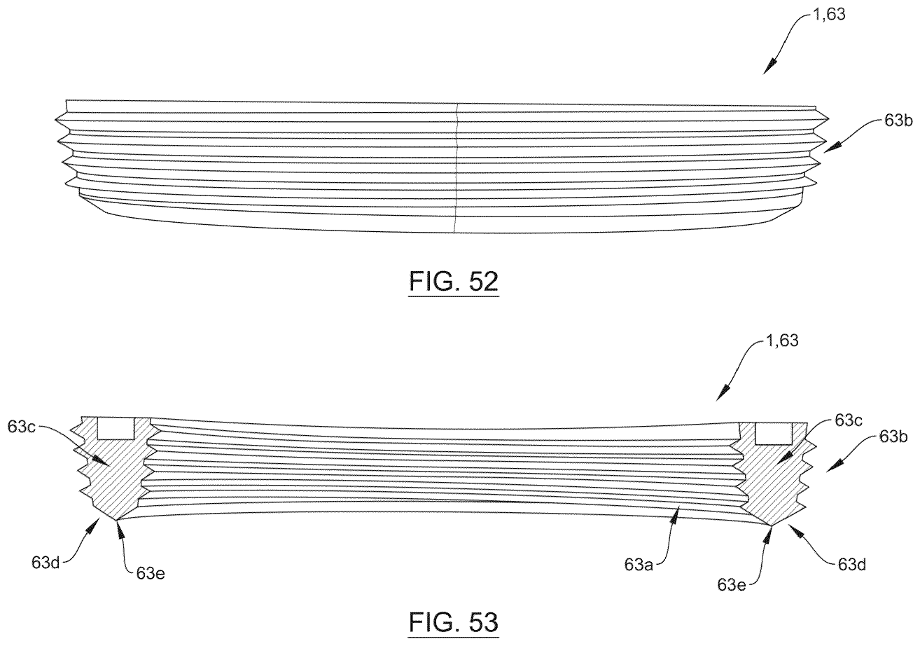

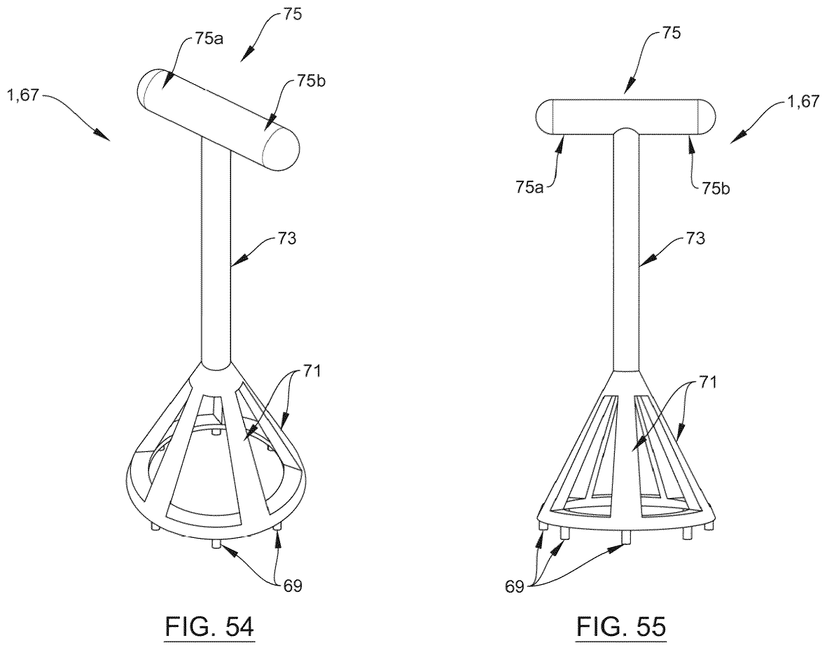

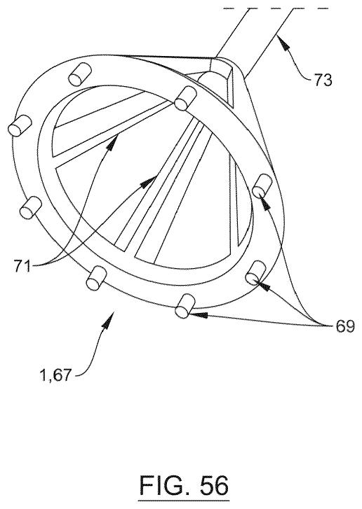

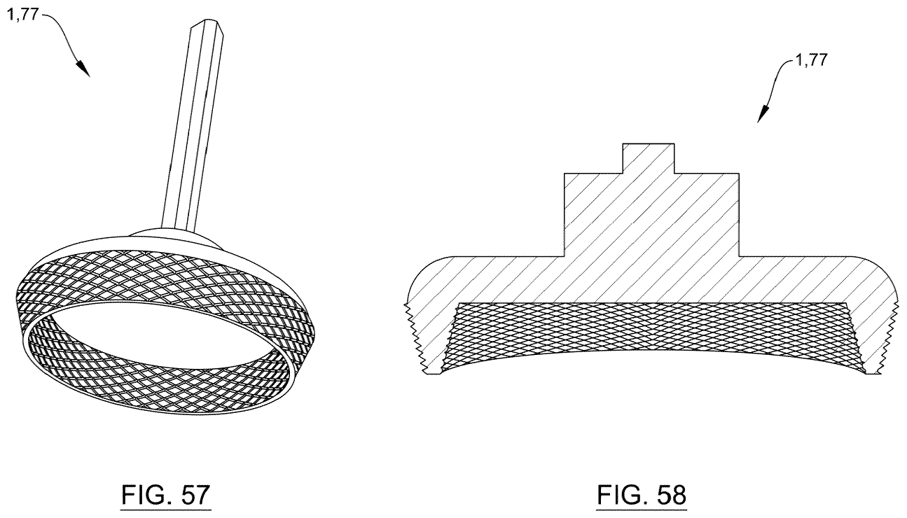

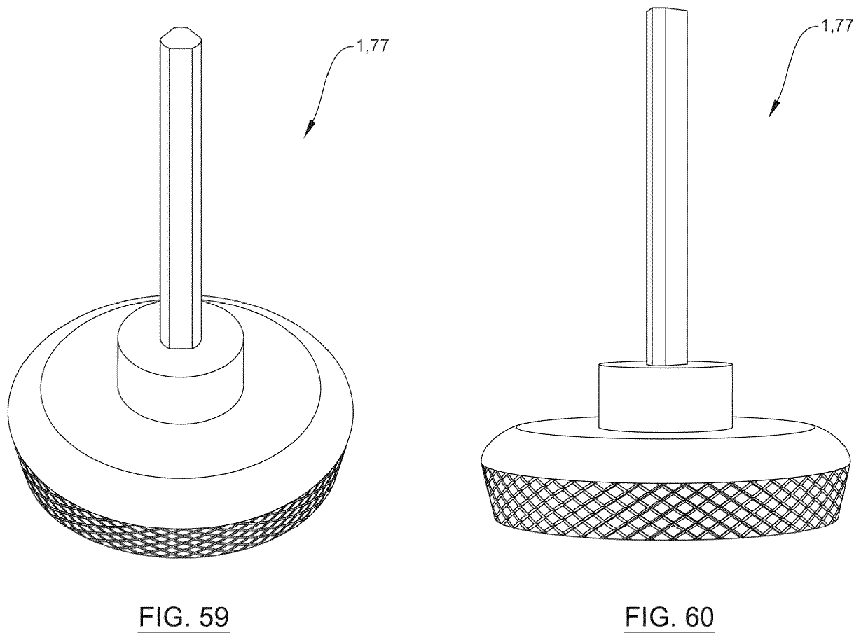

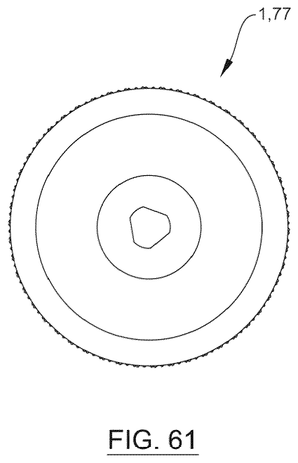

Therefore, for the connection system (ex. “axial compression screw system”) previously explained, instead of using an embodiment such as the one(s) described above and/or illustrated in FIGS. 12-26, for example, an “interfragmentary headless screw” system could be used to generate a compressive force at the exact site between two (2) elements that must be assembled, such as that exemplified in FIGS. 43-49, for example.

Namely, after having been cut vertically, the two (2) halves of the ball joint will be able to benefit from the advantages of this system. Indeed, the patella is a superficial bone lying underlying and in direct contact with the skin, with strong tendon attachments on either side of the medial and lateral edges. These characteristics oblige the particularity that the implant used, does not have a head because it would be embarrassing, conspicuous and most likely painful due to its prominence under the skin.

The principle of this “headless interfragmentary screw” is that in the absence of a screw head, the desired compression is generated by the difference in the grip in the bone between the two (2) halves of the patella. Thus, the side where the contact surface between the screw and the bone is smaller, slides in the direction of the side where the contact surface of the screw with the bone is larger. This is achieved by having two (2) different types of threads on each end of this screw-in other words, it is this difference between the two (2) threads that results in two (2) strengths of gripping into the bone, being “different”. All this allows the user (ex. operator, surgeon, etc.) to completely erase the free space between the two (2) patella fragments. It goes without saying that the orientation of the screw is as perpendicular as possible in relation to the cutting plane of the ball joint, and this, in the two (2) axes.

The central tunnel of this “headless interfragmentary screw” is present for the same reason as that already described in the first screw system where a pin with a diameter equal to that of the interior tunnel of the drill bit and the screw is positioned prior to the cut. During final assembly, this same pin will be returned exactly to the initial location, which facilitates guiding the screw through the drilled hole in question. This allows us to assemble the patella as perfectly as possible in relation to its initial condition, which is advantageous for various obvious reasons.

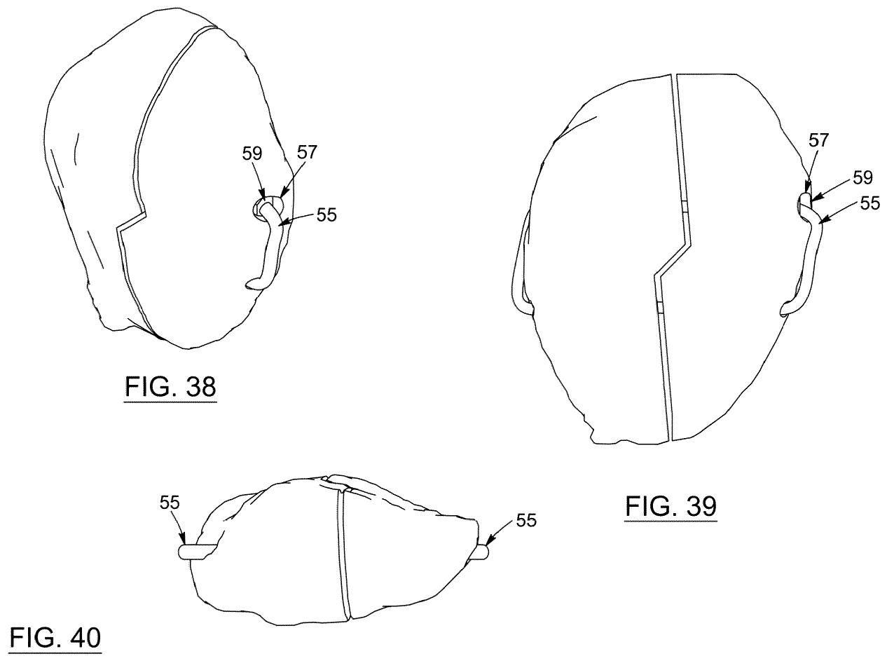

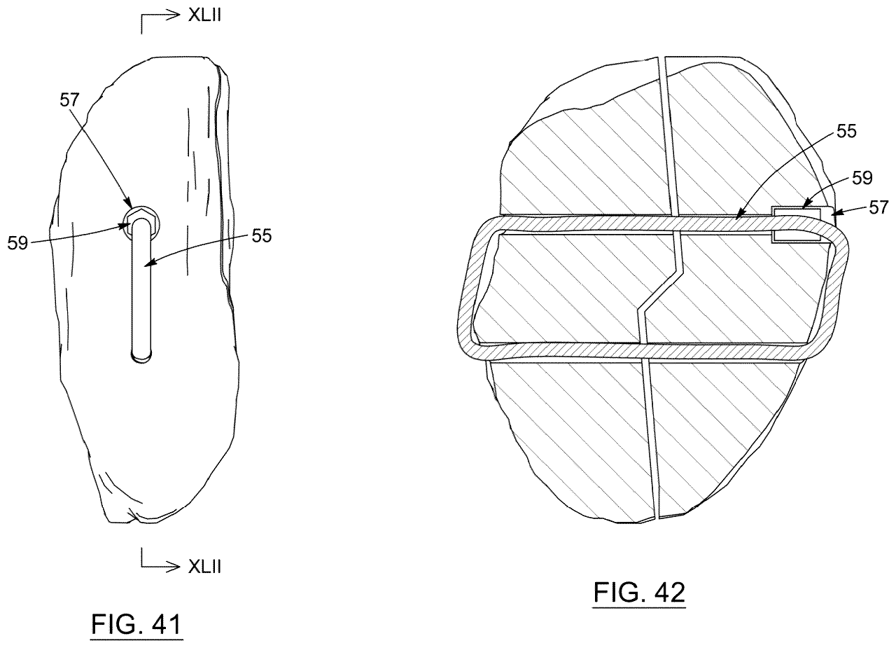

Alternatively, for the connection system (ex. “system for fixing the ball joint after having cut it”), one could also use a system (and/or a principle and/or a technique) of “cerclage” (eg. with tensioning by a tensioner and/or the like), such as that exemplified in FIGS. 38-42, for example. According to this particular embodiment being possible for the present system also, the technique of strapping (and/or winding, etc.) could use a wire (and/or a band, a link, etc.) of genius, resistant to a high tension, being braided with metallic filaments (for example), and being insertable into corresponding hole(s) and being secured in place, by a retention mechanism, such as a knot being closed and hidden (ex. recessed and/or in a hollow, etc.), for example, as illustrated in the corresponding figures attached.

As can now be better appreciated in view of the foregoing, the present invention is particularly advantageous insofar as it makes it possible to offer operating equipment comprising a series of new tools and/or accessories, in order to allow, in particular, a new surgical approach proposed hereby, namely trans-patellar bone and tendon with longitudinal cut, which could potentially remedy all the drawbacks encountered with the conventional surgical approach to knee arthroplasty, and which would be very most likely responsible for the poor results of this conventional intervention.

As can now also be better appreciated, the present invention is also advantageous insofar as it makes it possible to intervene surgically, on a joint, such as a knee, for example, and to be able to do so in a faster, simpler, more accurate, more efficient, more economical, more reliable, more tunable, more versatile, more adaptable, more durable, more desirable and/or improved manner than is possible with the current conventional way of doing things.

The present invention is also advantageous for all the various participants in the process (ex. patients, surgeons, operating teams, rehabilitation teams, insurance companies, health system, etc.).

Finally, and according to the present invention, the operating equipment (and/or resulting system, etc.) and its corresponding components are preferably made of substantially rigid materials, such as metallic materials (stainless steel, titanium, etc.), hardened polymers, composite materials, and/or any other suitable material(s), while the other components of the system according to the present invention, in order to obtain the advantages discussed above, can be made of any other suitable material(s), such as polymeric materials (plastic, rubber, etc.), and/or any other suitable material(s), depending on the particular application(s) for which the system is designed and the various parameters involved, as obvious to a person skilled in the art.

Although the present invention has been previously explained through preferred embodiments thereof, it should be understood that any modification to these preferred embodiments is not considered to change or alter the nature and scope of the present invention. Indeed, the attached claim(s) must not be limited in its (their) scope(s) by the preferred embodiments illustrated in the examples, but must receive the broadest interpretation that is consistent with the description as a whole.

1. An operating equipment (1) comprising at least one piece of equipment being positionable, shaped and sized to cooperate with a joint of a patient intended to be operated on, so as to allow a trans-patellar bone and tendon surgical intervention with longitudinal cut on the joint of the patient, via said at least one piece of equipment of the operating equipment.

2. An operating equipment (1) according to claim 1, wherein the operating equipment (1) comprises a sectioning guide (3) for sectioning into two sub-parts a bone part of the joint.

3. An operating equipment (1) according to claim 2, wherein the sectioning guide (3) comprises a groove (5) extending through a main body (7) of the sectioning guide (3), and defining a shape being intended to be used for severing the bone part of the joint.

4. An operating equipment (1) according to claim 3, wherein the groove (5) of the sectioning guide (3) is segmented, and comprises at least two different segments (5a,5b).

5. An operating equipment (1) according to claim 3, wherein the groove (5) of the sectioning guide (3) is segmented, and comprises at least three different segments (5a,5b,5c).

6. An operating equipment (1) according to claim 4 or 5, wherein each different segment (5a,5b,5c) from the groove (5) of the sectioning guide (3) is substantially rectilinear.

7. An operating equipment (1) according to claim 6, wherein each different rectilinear segment (5a,5b,5c) is connected to an adjacent segment (5a,5b,5c) via a transition segment (5d).

8. An operating equipment (1) according to any one of claims 3-7, wherein the groove (5) of the sectioning guide (3) comprises first and second distal segments (5a,5b).

9. An operating equipment (1) according to claim 8, wherein the groove (5) of the sectioning guide (3) comprises at least one intermediate segment (5c).

10. An operating equipment (1) according to any one of claims 4-9, wherein the different segments (5a,5b,5c,5d) define the groove (5) of the sectioning guide (3).

11. An operating equipment (1) according to any one of claims 3-10, wherein the groove (5) of the sectioning guide (3) defines a zigzag path.

12. An operating equipment (1) according to any one of claims 2-11, wherein the sectioning guide (3) comprises a recess (9) being positioned, shaped and sized in order to receive a frontal portion of the bone part of the joint.

13. An operating equipment (1) according to claim 12, wherein the recess (9) has a substantially elliptical contour (9c).

14. An operating equipment (1) according to any one of claims 2-13, wherein the sectioning guide (3) has at least one passage (11) for receiving a mounting means for selectively and temporarily mounting the sectioning guide (3) onto the frontal portion of the bone part of the joint being intended to be operated on surgically.

15. An operating equipment (1) according to any one of claims 2-14, wherein the sectioning guide (3) has at least one first side passage (11a) in a first side of the sectioning guide (3) to connect the sectioning guide (3) selectively and temporarily on a first side of the frontal portion of the bone part of the joint intended to be operated on surgically, and wherein the sectioning guide (3) comprises at least one second side passage (11b) on a second side of the sectioning guide (3) for connecting the sectioning guide (3) selectively and temporarily to a second side of the frontal portion of the bone part of the joint intended to be operated on surgically.

16. An operating equipment (1) according to any one of claims 2-15, wherein the sectioning guide (3) comprises at least a pair of first side passages (11a) and at least a pair of second side passages (11b), in order to provide the sectioning guide (3) with a minimum of four side passages (11, 11a, 11b).

17. An operating equipment (1) according to any one of claims 14-16, wherein each passage (11, 11a, 11b) is substantially cylindrical.

18. An operating equipment (1) according to any one of claims 14-17, wherein each passage (11, 11a, 11b) extends at an angle through the main body (7) of the sectioning guide (3).

19. An operating equipment (1) according to any one of claims 14-18, wherein each passage (11, 11a, 11b) is provided with a first surface end of the sectioning guide (3) and a second end in an internal part of the sectioning guide (3).

20. An operating equipment (1) according to claim 19, wherein the first end of each passage (11, 11a, 11b) comes from a different corner (13) from the sectioning guide (3).

21. An operating equipment (1) according to claim 20, wherein each corner (13) of the sectioning guide (3) is bevelled.

22. An operating equipment (1) according to any one of claims 19-21, wherein the second end of each passage (11, 11a, 11b) is directed towards a central portion of the sectioning guide (3).

23. An operating equipment (1) according to any one of claims 19-22, wherein the second end of each passage (11, 11a, 11b) opens into an internal portion of the sectioning guide (3).

24. An operating equipment (1) according to any one of claims 19-23, wherein the second end of each passage (11, 11a,11b) ends in a recess (9) of the sectioning guide (3) being designed to receive a front portion of the bone part of the joint intended to be operated on surgically.

25. An operating equipment (1) according to any one of claims 2-24, wherein the sectioning guide (3) comprises a main body (7) being substantially rectangular.

26. An operating equipment (1) according to claim 25, wherein the main body (7) of the sectioning guide (3) has a given thickness (15).

27. An operating equipment (1) according to claim 26, wherein the recess (9) being designed to receive a front portion of the bone part of the joint intended to be operated on surgically is housed inside the given thickness (15) of the main body (7) of the sectioning guide (3).

28. An operating equipment (1) according to any one of claims 2-27, wherein the operating equipment (1) comprises a sectioning tool (17) having a geometric profile complementary to a groove (5) of the sectioning guide (3), in order to allow an insertion of said sectioning tool (17) through said groove (5) of the sectioning guide (3).

29. An operating equipment (1) according to claim 28, wherein the sectioning tool (17) comprises a first cutting edge (17a) to allow cutting of the bone part of the joint into two corresponding sub-parts.

30. An operating equipment (1) according to claim 29, wherein the sectioning tool (17) comprises a second non-cutting edge (17b) in order to receive a tool for striking the sectioning tool (17) and thus allow the first cutting edge (17a) thereof to cut the bone part of the joint into two corresponding sub-parts.

31. An operating equipment (1) according to any one of claims 28-30, wherein the sectioning tool (17) has at least one lateral groove (17c).

32. An operating equipment (1) according to any one of claims 28-31, wherein the sectioning tool (17) comprises a pair of lateral grooves (17c).

33. An operating equipment (1) according to any one of claims 2-32, wherein the operating equipment (1) comprises a connection system (19) for reconnecting again and together the bone part of the joint having been sectioned into two sub-parts with the sectioning guide (3).

34. An operating equipment (1) according to claim 33, wherein the connection system (19) comprises at least one attachment (21) being insertable into a corresponding hole having been defined in the two corresponding sub-parts of the bone part of the joint having been severed, the attachment (21) being capable of being adjusted and tightened in order to bring the two sub-parts of the bone part of the joint together after the surgical intervention.

35. An operating equipment (1) according to claim 34, wherein said at least one attachment (21) comprises first and second attachments (21a,21b), at least one of these attachments (21a,21b) comprising at least one male part (23) intended to cooperate with at least one corresponding female part (25) of the other attachment (21b,21a), to allow corresponding interconnection and self-locking between said attachments (21a,21b).

36. An operating equipment (1) according to claim 35, wherein the first and second attachments (21a, 21b) each comprise a series of male and female parts (23,25) intended to cooperate with the corresponding female and male parts (25,23) of the other attachment (21b,21a).

37. An operating equipment (1) according to claim 35 or 36, wherein each attachment (21a,21b) is substantially “U” shaped, and comprises two sections (27) each being provided with a series of male and female parts (23,25) intended to cooperate with the corresponding female and male parts (25,23) of the other attachment (21b,21b).

38. An operating equipment (1) according to any one of claims 35-37, wherein each section (27) of each attachment (21a,21b) is provided with a series of male and female parts (23,25) being defined by alternating peaks and depressions.

39. An operating equipment (1) according to any one of claims 35-38, wherein each section (27) of each attachment (21a,21b) has a cross-section which decreases towards a distal end of said each section.

40. An operating equipment (1) according to any one of claims 33-39, wherein the connection system (19) comprises a first threaded head (29) being insertable into a first sub-part of the two sub-parts of the bone part of the joint having been severed with the sectioning guide (3), a second threaded head (31) being insertable into a second sub-part of the two sub-parts of the bone part of the joint having been severed, and a connection rod (33) cooperating with the two threaded heads (29,31), the connection system (19) being able to be adjusted and tightened by means of a rotation of the connection rod (33) in order to bring the two sub-parts of the bone part of the joint back together after surgery.

41. An operating equipment (1) according to claim 40, wherein the first threaded head (29) comprises an anchoring surface (29a) being provided with threading (35) in order to anchor this first threaded head (29) in the first sub-part of the two sub-parts of the bone part of the joint.

42. An operating equipment (1) according to claim 41, wherein the anchoring surface (29a) of the first threaded head (29) is tapered.

43. An operating equipment (1) according to any one of claims 40-42, wherein the first threaded head (29) is provided with a endpiece (29b) comprising at least one orifice (29c) for receiving a rotating and/or affixing tool for rotatively affixing and holding in place the first threaded head (29) in the first sub-part of the two sub-parts of the bone part of the joint.

44. An operating equipment (1) according to claim 43, wherein the endpiece (29b) of the first threaded head (29) has n corresponding orifices (29c), n being an integer greater than 1.

45. An operating equipment (1) according to claim 44, wherein the n orifices (29c) of the endpiece (29b) of the first threaded head (29) are identical with respect to each other.

46. An operating equipment (1) according to claim 44 or 45, wherein the n orifices (29c) of the endpiece (29b) of the first threaded head (29) are equidistant from one another.

47. An operating equipment (1) according to any one of claims 43-46, wherein the endpiece (29b) of the first threaded head (29) has 3 corresponding orifices (29c).

48. An operating equipment (1) according to any one of claims 43-47, wherein the endpiece (29b) of the first threaded head (29) has rounded peripheral contours (29d).

49. An operating equipment (1) according to any one of claims 43-48, wherein the endpiece (29b) of the first threaded head (29) has an axial hole (29e) being configured to receive a first end (33a) of the connecting rod (33).

50. An operating equipment (1) according to claim 49, wherein the axial hole (29e) of the first threaded head (29) is provided with an internal threading (35) configured to receive an external threading (35) corresponding to the first end (33a) of the connecting rod (33).

51. An operating equipment (1) according to any one of claims 40-50, wherein the second threaded head (31) comprises an anchoring surface (31a) being provided with threading (35) to anchor this second threaded head (31) in the second sub-part of the two sub-parts of the bone part of the joint.

52. An operating equipment (1) according to claim 50, wherein the anchoring surface (31a) of the second threaded head (31) is curved.

53. An operating equipment (1) according to any one of claims 40-52, wherein the second threaded head (31) is provided with an endpiece (31b) comprising at least one orifice (31c) for receiving a rotating and/or affixing tool for rotatively affixing and holding in place the second threaded head (31) in the second sub-part of the two sub-parts of the bone part of the joint.

54. An operating equipment (1) according to claim 53, wherein the endpiece (31b) of the second threaded head (31) has n corresponding orifices (31c), n being an integer greater than 1.

55. An operating equipment (1) according to claim 54, wherein the n orifices (31c) of the endpiece (31b) of the second threaded head (31) are identical with respect to each other.

56. An operating equipment (1) according to claim 54 or 55, wherein the n orifices (31c) of the endpiece (31b) of the second threaded head (31) are equidistant from one another.

57. An operating equipment (1) according to any one of claims 53-56, wherein the endpiece (31b) of the second threaded head (31) has 4 corresponding orifices (29c).

58. An operating equipment (1) according to any one of claims 53-57, wherein the endpiece (31b) of the second threaded head (31) has rounded peripheral contours (31d).

59. An operating equipment (1) according to any one of claims 53-58, wherein the endpiece (31b) of the second threaded head (31) comprises an axial hole (31e) being configured to receive a second end (33b) of the connecting rod (33).

60. An operating equipment (1) according to claim 59, wherein the axial hole (31e) of the second threaded head (31) is configured to leave a passage of the first end (33a) of the connecting rod (33), beforehand, so that this first end (33a) of the connecting rod (33) is connected on the first threaded head (29).

61. An operating equipment (1) according to any one of claims 40-60, wherein the connection rod (33) comprises a main rod (33c), a first end (33a) provided with threading (35) being complementary to a corresponding internal threading (35) of the axial hole (29e) of the first threaded head (29), and a second end (33b) provided with a corresponding head (33d) provided with a cavity to receive a corresponding rotating tool.

62. An operating equipment (1) according to any one of claims 40-61, wherein the operating equipment (1) comprises a corresponding first router bit (39) having a geometric profile being substantially complementary to that of the main body of the first threaded head (29), the first router bit (39) being used to drill a corresponding hole in the first sub-part of the two sub-parts of the bone part of the joint intended to receive the first threaded head (29).

63. An operating equipment (1) according to claim 62, wherein the operating equipment (1) comprises a corresponding second router bit (41) having a geometric profile being substantially complementary to that of the main body of the second threaded head (31), the second router bit (41) being used to drill a corresponding hole in the second sub-part of the two sub-parts of the bone part of the joint intended to receive the second threaded head (31).

64. An operating equipment (1) according to any one of claims 1-63, wherein the operating equipment (1) comprises a measuring tool (43) for measuring a distance relative to the corresponding hole to be defined in the two corresponding sub-parts of the bone part of the joint to be severed, in order to be able to carry out the surgical intervention and to be able to subsequently better bring the two sub-parts of the bone part of the joint back together after the ‘surgical intervention.

65. An operating equipment (1) according to claim 64, wherein the measuring tool (43) comprises two main sections (43a,43b) being movable relative to each other.

66. An operating equipment (1) according to claim 64 or 65, wherein the measuring tool (43) has two measuring ends (45a,45b) being movable relative to each other through a relative movement between the two main sections (43a,43b) of the measuring tool (43).

67. An operating equipment (1) according to any one of claims 64-66, wherein at least one main section (43a,43b) of the measuring tool (43) comprises a measuring scale (47) and a corresponding reading line (49), in order to be able to measure a distance between the two measuring ends (45a,45b) of the measuring tool (43).

68. An operating equipment (1) according to any one of claims 64-67, wherein each main section (43a,43b) of the measuring tool (43) comprises a measuring scale (47) and a corresponding reading line (49), in order to be able to measure a distance between the two measuring ends (45a,45b) of the measuring tool (43).

69. An operating equipment (1) according to any one of claims 64-68, wherein the measuring tool (43) comprises at least one main tightening component (51) in order to hold in place in a fixed manner, the two main sections (43a,43b), and therefore the two corresponding measuring ends (45a,45b), of the measuring tool (43), relative to each other.

70. An operating equipment (1) according to claim 69, wherein said at least one main tightening component (51) comprises a handle (53,53a,53b).

71. An operating equipment (1) according to claim 70, wherein the handle (53,53a,53b) of said at least one main tightening component (51) is rounded.

72. An operating equipment (1) according to claim 70 or 71, wherein the handle (53,53a,53b) of said at least one main tightening component (51) comprises a knurled peripheral surface in order to allow an increased manual grip by a user of the measuring tool (43).

73. An operating equipment (1) according to any one of claims 69-72, wherein said at least one main tightening component (51) comprises two main tightening components (51a,51b), one for each main section (43a,43b) of the measuring tool (43), in order to hold in place in a fixedly manner, the two main sections (43a,43b), and therefore, the two measuring ends (45a,45b), of the measuring tool (43), relative to each other.

74. An operating equipment (1) according to any one of claims 64-73, wherein the measuring tool (43) comprises at least one complementary section (43c,43d) being movable relative to a main body of the measuring tool (43), a measuring end (45,45a,45b) of the measuring tool (43) being mounted on said complementary section (43c,43d).

75. An operating equipment (1) according to any one of claims 64-74, wherein the measuring tool (43) comprises two complementary sections (43c,43d) each being movable relative to a main body of the measuring tool (43), a measuring end (45,45a,45b) of the measuring tool (43) being mounted on each of said complementary sections (43c,43d).

76. An operating equipment (1) according to claim 75, wherein at least one complementary section (43c,43d) of the measuring tool (43) comprises a measuring scale (47) and a corresponding reading line (49), in order to be able to measure a distance between a longitudinal axis (43e) of the measuring tool (43) and a corresponding measuring end (45,45a,45b).

77. An operating equipment (1) according to claim 75 or 76, wherein each complementary section (43c,43d) of the measuring tool (43) comprises a measuring scale (47) and a corresponding reading line (49), in order to be able to measure a distance between a longitudinal axis (43e) of the measuring tool (43) and a corresponding measuring end (45,45a,45b) associated with a given complementary section (43c,43d).

78. An operating equipment (1) according to any one of claims 64-77, wherein the measuring tool (43) comprises at least one complementary tightening component (51c,51d) in order to hold in place, at least one complementary section (43c,43d) and a corresponding measuring end (45,45a,45b), fixedly relative to a main body of the measuring tool (43).

79. An operating equipment (1) according to claim 78, wherein said at least one additional tightening component (51c, 51d) comprises a handle (53, 53c, 53d).

80. An operating equipment (1) according to claim 79, wherein the handle (53,53c,53d) of said at least one complementary tightening component (51c,51d) is rounded.

81. An operating equipment (1) according to claim 79 or 80, wherein the handle (53,53c,53d) of said at least one additional tightening component (51c,51d) comprises a knurled peripheral surface in order to allow an increased manual grip by a user of the measuring tool (43).

82. An operating equipment (1) according to any one of claims 79-81, wherein said at least one complementary tightening component (51c,51d) comprises two complementary tightening components (51c,51d), one for each complementary section (43c,43d) of the measuring tool (43), in order to hold in place, each complementary section (43c,43d) and a corresponding measuring end (45,45a,45b), fixedly relative to a main body of the measuring tool (43).

83. An operating equipment (1) according to any one of claims 33-82, wherein the connection system (19) comprises a strapping system.

84. An operating equipment (1) according to claim 83, wherein the strapping system is selectively tensioned by means of a tensioner.

85. An operating equipment (1) according to claim 83 or 84, wherein the strapping system comprises at least one wire (55).

86. An operating equipment (1) according to claim 85, wherein said at least one wire (55) of the strapping system comprises at least one high-tension wire (55).

87. An operating equipment (1) according to claim 85 or 86, wherein said at least one wire (55) of the strapping system comprises at least one wire metallic (55).

88. An operating equipment (1) according to any one of claims 85-87, wherein said at least one wire (55) of the strapping system comprises a plurality of metallic filaments being braided together.

89. An operating equipment (1) according to any one of claims 85-88, wherein said at least one wire (55) of the strapping system is insertable at least one corresponding hole (57) of the patella.

90. An operating equipment (1) according to any one of claims 85-89, wherein said at least one wire (55) of the strapping system is adapted to be fixed in place by a retention mechanism (59).

91. An operating equipment (1) according to any one of claims 33-90, wherein the connection system (19) comprises a headless interfragmentary screw system (61), having a longitudinal piece (61c), and first and second opposite pieces (61a,61b) with outer threaded surfaces, said first and second pieces (61a,61b) being mountable and movable along said longitudinal piece portion (61c).

92. An operating equipment (1) according to claim 91, wherein geometric disposition of the first and second opposite parts (61a,61b) are different from each other.

93. An operating equipment (1) according to claim 91 or 92, wherein each opposite piece (61a,61b) has a distal cavity (61d) being positioned, shaped and sized to receive a corresponding tool intended for screwing and/or unscrewing the first and second opposite pieces (61a,61b) in displacement relative to each other along the longitudinal part (61c) of the headless interfragmentary screw system (61).

94. An operating equipment (1) according to any one of claims 91-93, wherein the longitudinal piece (61c) of the headless interfragmentary screw system (61) comprises at least one longitudinal groove (611).

95. An operating equipment (1) according to any one of claims 91-94, wherein the longitudinal part (61c) of the headless interfragmentary screw system (61) comprises at least peripheral longitudinal groove (611) and/or at least one axial longitudinal groove (611).

96. An operating equipment (1) according to any one of claims 33-95, wherein the connection system (19) comprises a screwable ring (63).

97. An operating equipment (1) according to claim 96, wherein the screwable ring (63) has an internal peripheral part (63a) and an external peripheral part (63b).

98. An operating equipment (1) according to claim 96 or 97, wherein at least a peripheral part (63a,63b) of the screwable ring (63) is provided with a threaded surface.

99. An operating equipment (1) according to claim 97 or 98, wherein the two peripheral parts (63a,63b) of the screwable ring (63) are provided with respective threaded surfaces.

100. An operating equipment (1) according to any one of claims 96-99, wherein the screwable ring (63) comprises a main body (63c) being substantially circular.

101. An operating equipment (1) according to claim 100, wherein the main body (63c) of the screwable ring (63) has a cross-section being profiled.

102. An operating equipment (1) according to claim 100 or 101, wherein the cross-section of the main body (63c) of the screwable ring (63) has a distal portion (63d).

103. An operating equipment (1) according to claim 102, wherein the distal portion (63d) of the cross-section of the main body (63c) of the screwable ring (63) is substantially pointed.

104. An operating equipment (1) according to claim 102 or 103, wherein the distal portion (63d) of the cross-section of the main body (63c) of the screwable ring (63) has a distal rim (63e).

105. An operating equipment (1) according to any one of claims 102-104, wherein the distal rim (63e) of the cross-section of the main body (63c) of the screwable ring (63) is substantially sharp.

106. An operating equipment (1) according to any one of claims 100-105, wherein an upper part of the main body (63c) of the screwable ring (63) is provided with at least one corresponding cavity (65) to receive a rotating tool (67).

107. An operating equipment (1) according to claim 106, wherein the upper part of the main body (63c) of the screwable ring (63) is provided with corresponding 2n cavities (65) to receive the corresponding rotating tool (67), n being an integer being equal to and/or greater than 1.

108. An operating equipment (1) according to claim 107, wherein each corresponding cavity (65) of the screwable ring (63) is capable of receiving a corresponding protruding element (69) of the corresponding rotating tool (67).

109. An operating equipment (1) according to claim 107 or 108, wherein each corresponding cavity (65) of the screwable ring (63) is arranged around the main body (63c) of the screwable ring (63).

110. An operating equipment (1) according to any one of claims 107-109, wherein each corresponding cavity (65) of the screwable ring (63) is arranged equidistantly from one another around the main body (63c) of the screwable ring (63).

111. An operating equipment (1) according to any one of claims 107-110, wherein the upper part of the main body (63c) of the screwable ring (63) is provided with at least two corresponding cavities (65) to receive protruding elements (69) of the corresponding rotating tool (67).

112. An operating equipment (1) according to any one of claims 107-111, wherein the upper part of the main body (63c) of the screwable ring (63) is provided with at least four corresponding cavities (65) to receive protruding elements (69) of the corresponding rotating tool (67).

113. An operating equipment (1) according to any one of claims 107-112, wherein the upper part of the main body (63c) of the screwable ring (63) is provided with at least six corresponding cavities (65) to receive protruding elements (69) of the corresponding rotating tool (67).

114. An operating equipment (1) according to any one of claims 107-113, wherein the upper part of the main body (63c) of the screwable ring (63) is provided with at least eight corresponding cavities (65) to receive protruding elements (69) of the corresponding rotating tool (67).

115. An operating equipment (1) according to any one of claims 106-114, wherein the corresponding rotating tool (67) comprises a number of protruding elements (69) being complementary with the number of corresponding cavities (65) provided on the screwable ring (63).

116. An operating equipment (1) according to claim 115, wherein each protruding element (69) of the rotating tool (67) is complementary to at least one cavity (65) corresponding to the screwable ring (63).

117. An operating equipment (1) according to claim 115 or 116, wherein each protruding element (69) of the rotating tool (67) is complementary to each corresponding cavity (65) of the screwable ring (63).

118. An operating equipment (1) according to any one of claims 115-117, wherein each corresponding cavity (65) of the screwable ring (63) is substantially cylindrical, and therefore, wherein each protruding element (69) of the corresponding rotating tool (67) is also substantially cylindrical.

119. An operating equipment (1) according to any one of claims 115-118, wherein each protruding element (69) of the rotating tool (67) is disposed on a distal portion of the rotating tool (67).

120. An operating equipment (1) according to claim 119, wherein the distal part of the rotating tool (67) is substantially circular and complementary to the upper part of the screwable ring (63).

121. An operating equipment (1) according to claim 119 or 120, wherein the distal part of the rotating tool (67) is provided with reinforcing parts (71) converging into a common rod (73).

122. An operating equipment (1) according to any one of claims 106-121, wherein the rotating tool (67) is provided with a proximate manipulation portion (75).

123. An operating equipment (1) according to claim 122, wherein the proximate manipulation portion (75) is provided on a common rod (73) of the rotating tool (67).

124. An operating equipment (1) according to claim 122 or 123, wherein the proximate manipulation portion (75) comprises a pair of opposite handles (75a,75b).

125. An operating equipment (1) according to any one of claims 96-124, wherein the operating equipment (1) comprises a grinding tool (77) having a geometric profile substantially complementary to that of the body main (63c) of the screwable ring (63), in order to enable a corresponding circular depression to be hollowed out beforehand in the patella, being intended to receive said screwable ring (63).
